(19) United States
(12) Patent Application Publication
(10) Pub. No.: US 2008/0293740 A1
(43) Pub. Date: Nov. 27, 2008

(54) METHOD OF TREATING ACID-SENSING ION CHANNEL MEDIATED PAIN, COUGH SUPPRESSION, AND CENTRAL NERVOUS SYSTEM DISORDERS

(75) Inventors: Michael R. JOHNSON, Chapel Hill, NC (US); Richard C. BOUCHER, Chapel Hill, NC (US); Andrew J. HIRSH, Durham, NC (US)

Correspondence Address: OBLON, SPIVAK, MCCLELLAND MAIER & NEUSTADT, P.C., 1940 DUKE STREET, ALEXANDRIA, VA, 22314, US

(73) Assignee: PARION SCIENCES, Inc., Durham, NC (US)

(21) Appl. No.: 12/061,837

(22) Filed: Apr. 3, 2008

Related U.S. Application Data
(60) Provisional application No. 60/909,802, filed on Apr. 3, 2007.

(51) Int. Cl. A61K 31/4965; A61P 25/00

(52) U.S. Cl. 514/255.06

(57) ABSTRACT

The present invention provides a variety of methods of treatment of acid-sensing ion channel (ASIC) mediated pain, cough, and central nervous system disorders by ASICs inhibition with a series of pyrazinoylguanidine compounds represented by formula (I) as defined herein.

### CONTINUING APPLICATION DATA

This application claims priority to U.S. application Ser. No. 60/909,802, filed on Apr. 3, 2007, and incorporated herein by reference in its entirety.

### BACKGROUND OF THE INVENTION

1. Field of the Invention

The present invention relates to acid-sensing ion channel blockers. The present invention provides a variety of methods of treatment of acid-sensing ion channel (ASIC) mediated pain, cough, and central nervous system disorders by ASICs inhibition with a series of pyrazinoylguanidine compounds represented by formula (I) as defined herein.

2. Description of the Background

In a lifetime, an individual will experience acute or chronic pain to some degree. Pain can arise from traumatic injuries, oropharangeal diseases or damage, tissue inflammation or infection, angina, stroke, ischemic heart disease, arthritis, cancer, gastrointestinal disorders, etc. In order to relieve pain, doctors prescribe drugs such as ibuprofen, a nonsteroidal anti-inflammatory drug (NSAID) or analgesics such as acetaminophen or aspirin, which are among the most frequently used in the United States. A major side-effect of these pain medications is a moderate (up to 38%) increase in developing high blood pressure. A novel approach to relieve pain and possibly decrease the incidence of developing high blood pressure is by directly blocking a proposed cellular protein involved in the pathway for nociceptor signal transduction, the acid-sensing ion channel (ASIC).

The ASIC represents an hydrogen-gated subgroup of channels in the degenerin/epithelial sodium channel family. Similar to the epithelial sodium channel, ASICs are also blocked by the potassium-sparing diuretic amiloride (Waldmann et al. Nature 1997), a novel synthetic chemical entity A-317567 (Dube et al. Pain 2005), a sea anemone peptide APETx2 (Diochot et al. Embro J. 2004) and a tarantula peptide toxin PcTX1 (Escoubas et al. 2000). The ASICs are prominent in both the peripheral and central nervous system, and to date comprised of six discrete subunits ASIC 1A, ASIC 1B, ASIC 2A, ASIC 2B, ASIC 3 and ASIC 4. The role of peripherally located ASICs are emerging as the main receptor for extracellular protons responding to tissue acidosis. One other protein activated by acid is the transient receptor potential vanilloid receptor (TRPV1). Chronic pain conditions associated with tissue acidosis include traumatic injuries, oropharangeal diseases or damage, tissue inflammation or infection, angina, stroke, ischemic heart disease, arthritis, cancer, and gastrointestinal disorders such as gastroesophageal reflux leading to heartburn.

The deep somatic pain originating in joints and tendons found in arthritis is a major therapeutic challenge. Spontaneous pain can develop as a consequence of sensitization of primary afferents directly involved in the inflammatory process, but also following sensitization of neuronal processing in the spinal cord (central sensitization) or higher centres. Inflammatory pain is linked to sensitization of sensory proteins at the nociceptive endings whereas pain originating from nerve damage has been linked to changes in axonal ion channels producing ectopic discharge in nociceptors as a source of pain. The ASIC3 is highly expressed on sensory neurons that innervate heart and skeletal muscle and is proposed to detect lactic acidosis and to transduce angina and muscle ischemic pain. Oropharangeal pain from disease or damage is likely mediated by ASIC3. ASIC3 neurons, which have large myelinated axons, are associated to the trigeminal ganglion neurons that supply the tooth pulp and facial skin with unmyelinated or finely myelinated axons. Inhibition of ASIC3 could relieve pain originating from tooth pulp and other areas of the mouth.

Stroke affects nearly four out of five Americans and is the number one cause of adult disability, leaving two of every three survivors with significant physical and emotional disabilities. Unfortunately, no effective therapeutic intervention for stroke-induced neural damage is available other than the use of short-acting thrombolytics, which have the potential side effect of intracranial hemorrhage. Also, the absence of a neuroprotective therapy became apparent following the failure of multiple clinical trials using glutamate antagonists as therapeutic agents. ASIC blockers would be a new therapy that could provide relief due to stroke.

In the central nervous system, ASICs are linked to learning and memory function as well as fear related behavior. The ASIC1 is found to contribute to synaptic plasticity in the hippocampus and to hippocampus-dependent spatial memory. ASIC1 is present in the hippocampal circuit, and more abundant in several areas outside the hippocampus (glomerulus of the olfactory bulb, whisker barrel cortex, cingulate cortex, striatum, nucleus accumbens, amygdala, and cerebellar cortex). As examples of the effect of ASIC in the central nervous system 1) an extracellular acidosis in amygdala neurons elicites a greater current density than hippocampal neurons and 2) disrupting the ASIC1 gene eliminated H+-evoked currents in the amygdala. The ASIC1 distribution in the central nervous system supports high levels of synaptic plasticity and contributes to the neural mechanisms of fear conditioning. Acidosis is a common feature of ischemic brain, and has been suggested to play a role in neuronal injury. In the central nervous system neurons, lowering extracellular pH to the level commonly seen in ischemic brain activates inward ASIC currents resulting in membrane depolarization. Blockade of ASIC1a inhibits the acid-induced currents, membrane depolarization, and in the end neuronal injury. In focal ischemia, ASIC1a blockade, or ASIC1a gene knockout both protect brain from injury. The blockers of ASIC1a also demonstrate a prolonged therapeutic window, beyond that of the glutamate antagonists.

Acid is also an important mediator in the pathogenisis of cough. Cough is the single most common symptom prompting outpatient medical visits in the United States, accounting for 20 million office visits in 1999 (2.7% of the total number of visits). The prevalence of cough depends on smoking status, and cough prevalence has been estimated at 5% to 40%, depending on the group studied. The aggregate cost of treatment alone for cough exceeds $1 billion in the United States. This cost is in addition to resources expended for repeated diagnostic studies. Acid directly stimulates vagal bronchopulmonary sensory nerves that regulate the cough reflex, by blocking ASIC and decreasing the acid responsible for neural stimulation the cough reflux pathway would potentially undergo inhibition. Cough is an important physiologic defense mechanism, a protective reflex to augment the mucociliary clearance of airway secretions. The cough reflex is characterized by the generation of high intrathoracic pressures against a closed glottis, followed by forceful expulsion of air and secretions on glottic opening. The symptom of cough involves a reflex arc originating in peripheral cough receptors.

Cough receptors are most concentrated in the epithelium of the upper and lower respiratory tracts, but are also located in the external auditory meatus, tympanic membrane, esophagus, stomach, pericardium, and diaphragm. Receptors are predominantly of two types. Irritant receptors are stimulated by noxious fumes or liquids, while mechanical receptors are activated by physical triggers such as touch, displacement, or stretch. Signals from the receptors are carried by vagal afferents to a medullary cough center, which then triggers cough activation via efferents mediated by the vagal, phrenic, and spinal motor nerves. Cough modulation is partly under the control of cortical stimuli. Therefore, irritation anywhere along the reflex arc by a disease process can cause cough.

A cough can be classified as acute (<3 to 8 weeks) or chronic/persistent. Most of the attention by clinicians is devoted to the chronic/persistent variety, since this is the variety that usually prompts patients to seek medical care. Postnasal drainage is the single most common cause of chronic cough, accounting for 8% to 87% of cases, either exclusively or in combination with other factors. Asthma is the second most common cause of chronic cough in adults, present in 14% to 55% of cases. Gastroesophageal reflux disease (GERD) accounts for up to 40% of chronic cough. It has been recognized as a contributor to cough with increasing frequency in observational studies; indeed, in recent investigations, it has often surpassed other causes of chronic cough. GERD frequently accompanies other causes of cough) i.e up to 80% of asthmatic patients have abnormal 24-hour pH probe findings. Recurrent elevations in abdominal pressure may contribute to this phenomenon. A self-perpetuating cycle of cough and GERD may ensue, making identification and treatment of GERD crucial in the integrated management of all cough syndromes.

Angiotensin-converting enzyme normally degrades proinflammatory mediators such as bradykinins and substance P. Inhibition of this action lowers the threshold for cough sensitivity. Cough due to ACE inhibitors is a class effect and has been documented with all ACE inhibitors in use; switching to another agent will not ameliorate the symptoms.

Chronic bronchitis (CB) is characterized by a productive cough on most days for 3 months in 2 consecutive years. It may be caused by irritant-induced inflammation or by the need to mobilize excessive secretions. Although CB is a frequent cause of cough in the population, it is present in only 5% of those seeking medical attention for cough. Cigarette smoke is the most common irritant, but occupational exposures or inflammatory bowel disease may also trigger this syndrome.

### SUMMARY OF THE INVENTION

It is an object of the present invention to provide compounds that block ASICs to treat peripheral nervous system pain, cough, and central nervous system disorders.

The compounds of Formula I, which have been found to be potent inhibitors of ASIC, provide a therapeutic pharmacodynamic half-life on the ASICs channel.

It is the object of the present invention to provide compounds for treatment that take advantage of the pharmacological properties of the compounds described above.

In particular, it is an object of the present invention to provide compounds for treatment which rely on blockade of ASIC to alleviate pain, cough, and central nervous system disorders.

It is another object of the present invention to provide compounds that target ischemic pain.

In particular, it is an object of the present invention to provide compounds for treating ischemic pain due to cardiovascular disease.

In particular, it is an object of the present invention to provide compounds for treating stroke-induced neural damage.

In particular, it is an object of the present invention to provide compounds for treating pain due to arthritis.

In particular, it is an object of the present invention to provide compounds for treating ischemic pain due to cancer.

In particular, it is an object of the present invention to provide compounds for treating pain due to inflammation.

In particular, it is an object of the present invention to provide compounds for treating pain due to infection.

In particular, it is an object of the present invention to provide compounds for treating pain due to oropharengeal diseases or damage.

In particular, it is an object of the present invention to provide compounds for treating ischemic pain due to traumatic injuries.

In particular, it is an object of the present invention to provide compounds for treating acute and chronic cough.

In particular, it is an object of the present invention to provide compounds for treating pain due to gastrointestinal disorders including GERD leading to chronic heartburn.

In particular, it is an object of the present invention to provide compounds for treating central nervous system disorders and psychiatric diseases or manifestations such as memory loss, learning disabilities, fear and anxiety.

It is the object of the present invention to provide methods of treatment that take advantage of the pharmacological properties of the compounds described above.

The object of the present invention may be accomplished with a class of pyrazinoylguanidine compounds represented by formula (I):

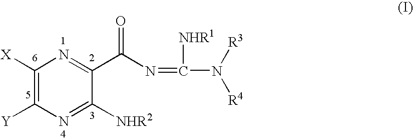

wherein the definition of these compounds and the parameters of R₁, R², R³, R⁴, X any Y are found in the variously defined pyrazinoylguanidine compounds described in U.S. Pat. No. 6,858,614, Feb. 22, 2005; U.S. Pat. No. 6,858,615, Feb. 22, 2005, U.S. Pat. No. 6,903,105, Jun. 7, 2005; U.S. Pat. No. 6,995,160 , Feb. 7, 2006; U.S. Pat. No. 7,026,325, Apr. 11, 2006; U.S. Pat. No. 7,030,117, Apr. 18, 2006; U.S. Pat. No. 7,064,129, Jun. 20, 2006; U.S. patent Ser. No. 10/828,171; U. S. patent Ser. No. 10/828,352; patent Ser. No. 61/031,466; U. S. patent Ser. No. 10/828,466; U. S. patent Ser. No. 10/828,278, and the following Published U.S. patent applications:

1. US Patent Application Publication # US2004/0229884A1, Nov. 18, 2004

2. US Patent Application Publication # US2004/0204425A1, Oct. 14, 2004

3. US Patent Application Publication # US2004/0204424A1, Oct. 14, 2004

4. US Patent Application Publication # US2004/0198749A1, Oct. 7, 2004

5. US Patent Application Publication # US2004/0198748A1, Oct. 7, 2004

6. US Patent Application Publication # US2004/0198747A1, Oct. 7, 2004

7. US Patent Application Publication # US2004/0198746A1, Oct. 7, 2004

8. US Patent Application Publication # US2004/0198745A1, Oct. 7, 2004

9. US Patent Application Publication # US2004/0198744A1, Oct. 7, 2004

10. US Patent Application Publication # US2004/0162296A 1, Aug. 19, 2004

11. US Patent Application Publication # US2003/0199456A1, Oct. 23, 2003

12. US Patent Application Publication # US2003/0195160A1, Oct. 16, 2003

13. US Patent Application Publication # US2005/059676A1, March 17, 2005.

14. US Patent Application Publication # US2005/0080091A1,Apr. 14, 2005.

15. US Patent Application Publication # US2005/00800921A1,Apr. 14, 2005.

16. US Patent Application Publication # US2005/0090505A1,Apr. 28, 2005.

17. US Patent Application Publication # US2005/0113390A1, May 26, 2005.

18. US Patent Application Publication # US2005/0113389A1, May 26, 2005.

19. US Patent Application Publication # US2005/0113388A1, May 26, 2005.

20. US Patent Application Publication # US2005/0080093A1, Apr. 14, 2005.

21. US Patent Application Publication # US2005/0228182A1, Oct. 13, 2005

22. US Patent Application Publication # US2005/0234072A1, Oct. 20, 2005

23. US Patent Application Publication # US2006/0040954A1, Feb. 23, 2006.

24. US Patent Application Publication # US2006/0052394A1, Mar. 9, 2006.

25. US Patent Application Publication # US2006/0052395A1, Mar. 9, 2006.

26. US Patent Application Publication # US2006/0063780 A1, Mar. 23, 2006.

27. US Patent Application Publication # US2006/0142306 A1, Jun. 29, 2006.

28. US Patent Application Publication # US2006/0142581 A1, Jun. 29, 2006.

29. US Patent Application Publication # US2006/0205738 A1, Sep. 9, 2006.

30. US Patent Application Publication # US2007/0021439 A1, Jan. 25, 2007.

31. US Patent Application Publication # US2007/0032509 A1, Feb. 8, 2007.

Each of the applications and patents cited above is incorporated herein by reference.

The compounds of Formula I described above can be a pharmaceutically acceptable salt thereof, and wherein the above compounds are inclusive of all racemates, enantiomers, diastereomers, tautomers, polymorphs and pseudopolymorphs thereof.

Each of the patents and applications cited above are incorporated herein by reference in their entirety, inclusive of specific compounds described therein.

The present also provides pharmaceutical compositions which contain a compound of Formula I described above.

The present invention also provides compounds of Formula I and the method of alleviating ASIC mediated pain, cough, and central nervous system disorders comprising:

administering an effective amount of a compound represented by formula (I) to a mucosal surface of a subject.

In particular, the present invention provides the following embodiments:
topically administering an effective amount of compound represented by formula (I) to the tissue of a subject,
er os administering an effective amount of compound represented by formula (I) to the tissue of a subject, and
intravenous administrating an effective amount of compound represented by formula (I) to the tissue of a subject.

The present invention also provides a method of inhibiting ASIC channels, comprising:

contacting and blocking ASIC channels with an effective amount of a compound represented by formula (I).

The present invention also provides a method of treating pain due to tissue ischemia, comprising administering the compound represented by formula (I) to the tissue of a subject.

The present invention also provides a method of treating ischemic pain.

The present invention also provides a method of treating pain due to cardiovascular disease.

The present invention also provides a method of treating stroke-induced neural damage.

The present invention also provides a method of treating pain due to arthritis.

The present invention also provides a method of treating ischemic pain due to cancer.

The present invention also provides a method of treating pain due to inflammation.

The present invention also provides a method of treating pain due to infection.

The present invention also provides a method of treating pain due to oropharengeal diseases or damage.

The present invention also provides a method of treating pain ischemic pain due to traumatic injuries.

The present invention also provides a method of treating chronic cough and cough associated with gastro-oesophageal reflux.

The present invention also provides a method of treating pain due to gastrointestinal disorders including GERD leading to chronic heartburn.

The present invention also provides methods of treating central nervous system disorders and psychiatric diseases or manifestations such as memory loss, learning disabilities, fear and anxiety.

### DETAILED DESCRIPTION OF THE INVENTION

The present invention is based on the discovery that the compounds of formula (I) are more potent blockers of ASICs.

The present invention is also based on the discovery that certain compounds embraced by Formula I target cardiovascular/ischemic pain.

The present invention is also based on the discovery that certain compounds embraced by Formula I target ischemic pain due to stroke.

The present invention is also based on the discovery that certain compounds embraced by Formula I target pain due to arthritis.

The present invention is also based on the discovery that certain compounds embraced by Formula I target ischemic pain due to cancer.

The present invention is also based on the discovery that certain compounds embraced by Formula I target pain due to inflammation.

The present invention is also based on the discovery that certain compounds embraced by Formula I target pain due to infection.

The present invention is also based on the discovery that certain compounds embraced by Formula I target ischemic pain due to traumatic injuries.

The present invention is also based on the discovery that certain compounds embraced by Formula I target pain due to oropharengeal diseases or damage.

The present invention is also based on the discovery that certain compounds embraced by Formula I target chronic cough and cough associated with gastro-oesophageal reflux.

The present invention is also based on the discovery that certain compounds embraced by Formula I target pain due to gastrointestinal disorders including GERD leading to chronic heartburn.

The present invention is also based on the discovery that certain compounds embraced by Formula I target central nervous system disorders and psychiatric diseases or manifestations such as memory loss, learning disabilities, fear and anxiety.

The compounds of formula I may be represented as:

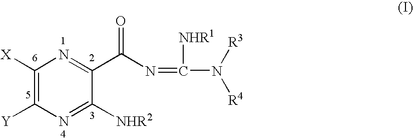

and racemates, enantiomers, diastereomers, tautomers, polymorphs, pseudopolymorphs and pharmaceutically acceptable salts, thereof, wherein:

X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;

Y is hydrogen, hydroxyl, mercapto, lower alkoxy, lower alkyl-thio, halogen, lower alkyl, unsubstituted or substituted mononuclear aryl, or —N(R²)₂;

R¹ is hydrogen or lower alkyl;

each R² is, independently, —R⁷, (CH₂)ₘ, —OR⁸, —(CH₂)ₘ, —NR⁷R¹⁰, —(CH₂)ₙ(CHOR⁸ )(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ—(Z)_{g}—R⁷, —(CH₂)ₘ—N¹⁰—CH₂(CHOR⁸)(CHOR⁸)ₙ—CH₂OR⁸, —(CH₂)ₙ—CO₂R⁷, or

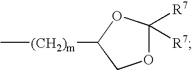

R³ and R⁴ are each, independently, hydrogen, lower alkyl, hydroxyl-lower alkyl, phenyl, (phenyl)-lower alkyl, (halophenyl)-lower alkyl, ((lower-alkyl)phenyl)-lower-alkyl, ((lower-alkoxy)phenyl)-lower-alkyl, (naphthyl)-lower-alkyl, or (pyridyl)-lower-alkyl, or a group represented by formula A or formula B, with the proviso that at least one of R³ and R⁴ is a group represented by the formula A or formula B;

—(C(R^{L})₂)ₒ-x-(C(R^{L})₂)ₚA¹ formula A:

—(C(R^{L})₂)ₒ-x-(C(R^{L})₂)ₚA² formula B:

A¹ is a C₆-C₁₅-membered aromatic carbocycle substituted with at least one R⁵ and the remaining substituents are R⁶;

A² is a six to fifteen-membered aromatic heterocycle substituted with at least one R⁵ and the remaining substituents are R⁶ wherein said aromatic heterocycle comprises 1-4 heteroatoms selected from the group consisting of O, N, and S;

each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}—R⁷, —O—(CH₂)ₘ-(Z)_{g}—R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

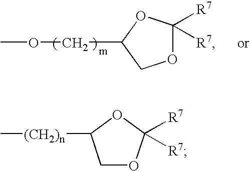

each o is, independently, an integer from 0 to 10;

each p is, independently, an integer from 0 to 10;

with the proviso that the sum of o and p in each contiguous chain is from 1 to 10;

each x is, independently, O, NR¹⁰, C(═O), CHOH, C(═N—R¹⁰), CHNR⁷R¹⁰, or a single bond;

each R⁵ is, independently, OH, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷,—OSO₃H, —O-glucuronide, —O-glucose,

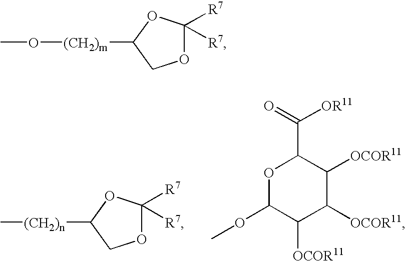

—(CH₂)ₙ—CO₂R¹³, -Het-(CH₂)ₘ—CO₂R¹³, —(CH₂)ₙ-(Z)_{g}-CO₂R¹³, -Het-(CH₂)ₘ,-(Z)_{g}-CO₂R¹³, —(CH₂)ₙ—NR¹⁰—(CH₂)ₘ(CHOR⁸)ₙ—CO₂R¹³, -Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, -Het-(CH₂)ₘ—(CHOR⁸)ₘ—CO₂R¹³, —(CH₂)ₙ—(CHOR⁸)ₘ(Z)_{g}-CO₂R¹³, -Het-(CH₂)ₙ—(CHOR⁸)ₙ-(Z)_{g}-CO₂R¹³, —(CH₂)ₙ-(Z)_{g}-(CH₂)ₘ—CO₂R¹³, —(CH₂)ₙ-(Z)_{g}-(CH₂)ₘ—CO₂R¹³, —(CH₂)ₙ-(Z)_{g}(CHOR⁸)ₘ-(Z)_{g}-CO₂R¹³, -Het-(CH₂)ₙ-(Z)_{g}-(CHOR⁸),ₙ-(Z)_{g}-CO₂R¹³, —(CH₂)ₙ—CONH—C(═NR¹³)—NR¹³R¹³, -Het-(CH₂)ₙ—CO—NH—C(═NR¹³)—NR¹³R¹³, —(CH₂)ₙ-(Z)_{g}-CONH—C(═NR¹³)—NR¹³R¹³, -Het-(CH₂)ₙ-(Z)_{g}-CONH—C(═NR¹³)—NR¹³R¹³, -Het-(CH₂)ₙ—NR¹⁰—(CH₂)ₙ—NR¹⁰—(CH₂)ₘ(CHOR⁸)ₙ—CONH—C(═NR¹³)—NR¹³R¹³, -Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, -Het-(CH₂)ₙ—(CHOR⁸)ₘ—CONH—C(═NR¹³)—NR¹³R^{—(CH}₂)ₙ—(CHOR⁸)ₘ-(Z)_{g}-CONH—C(═NR¹³)—NR¹³R¹³, -Het-(CH₂)ₙ—(CHOR⁸)ₘ-(Z)_{g}-CONH—C(═NR¹³)—NR¹³R¹³, —(CH₂)ₙ-(Z)_{g}-(CH₂)ₘCONH—C(═NR¹³)—NR¹³R¹³, -Het-(CH₂)ₙ-(Z)_{g}-(CH₂)ₘCONH—C(═NR¹³)—NR¹³R¹³, —(CH₂)ₙ-(Z)_{g}-(CHOR⁸)ₘ-(Z)_{g}-CONH—C(═NR¹³)—NR¹³R¹³, Het—(CH₂)ₙ-(Z)_{g}-(CHOR⁸)ₘ-(Z)_{g}-CONH—C(═NR )—NR¹³R¹³, —(CH₂)ₙ—CONR⁷—CONR¹³R¹³, -Het-(CH₂)ₙ—CONR⁷-CONR¹³R¹³, —(CH₂)ₙ-(Z)_{g}-CONR⁷-CONR¹³R¹³, —(CH₂)ₙ-(Z)_{g}-CONR⁷—CONR¹³R¹³, —(CH₂)ₙ—NR¹⁰—(CH₂)ₘ(CHOR⁸)ₙ—CONR⁷—CONR¹³R¹³, -Het-(CH₂)ₙ—NR¹⁰—(CH₂)ₘ(CHOR⁸)ₙ—CONR⁷—CONR¹³R¹³, —(CH₂)ₙ—(CHOR⁸)ₘ—CONR⁷-CONR¹³R¹³, -Het-(CH₂)ₘ—(CHOR⁸)ₘ—CONR⁷—CONR¹³R¹³, —(CH₂)ₙ—(CHOR⁸)ₘ-(Z)_{g}-CONR⁷—CONR¹³R¹³, -Het-(CH₂)ₙ—(CHOR⁸)ₘ-(Z)_{g}-CNR⁷—CONR¹³R¹³, —(CH₂)ₙ-(Z)_{g}-(CH₂)ₘCONR⁷—CONR¹³R¹³, -Het-(CH₂)ₙ-(Z)_{g}-(CH₂)ₘCONR⁷—CONR¹³R¹³, —(CH₂)ₙ-(Z)_{g}(CHOR⁸)ₘ-(Z)_{g}-CONR⁷—CONR¹³R¹³, -Het-(CH₂)ₙ-(Z)_{g}(CHOR⁸)ₘ-(Z)_{g}-CONR⁷—CONR¹³R¹³—(CH₂)ₙ—CONR⁷SO₂NR¹³R¹³, -Het-(CH₂)ₘ—CONR⁷SO₂NR¹³R¹³, —(CH₂)ₙ-(Z)_{g}-CONR⁷SO₂NR¹³R¹³, -Het-(CH₂)ₘ-(Z)_{g}-CONR⁷SO₂NR¹³R¹³, —(CH₂)ₙ—NR —(CH₂)ₘ(CHOR⁸)ₙ—CONR⁷SO₂NR¹³R¹³, -Het-(CH₂)ₘ-NR¹⁰—(CH₂)ₘ(CHOR⁸)ₙ—CONR⁷SO₂NR¹³R¹³, —(CH₂)ₙ—(CHOR⁸)ₘ—CONR⁷SO₂NR¹³R¹³, -Het-(CH₂)ₘ—(CHOR⁸)ₘ—CONR⁷SO₂NR¹³R¹³, —(CH₂)ₙ—(CHOR⁸)ₘ-(Z)_{g}-CONR⁷SO₂NR¹³R¹³, -Het-(CH₂)ₙ—(CHOR⁸)ₘ-(Z)_{g}-CONR⁷SO₂NR¹³R¹³, —(CH₂)ₙ-(Z)_{g}-(CH₂)ₘCONR⁷SO₂NR¹³R¹³, -Het-(CH₂)ₙ-(Z)_{g}-(CH₂)ₘCONR⁷SO₂NR¹³R¹³, —(CH₂)ₙ-(Z)_{g}-(CHOR⁸)ₘ-(Z)_{g}-CONR⁷SO₂N¹³R¹³, -Het-(CH₂)ₙ-(Z)_{g}-(CHOR⁸)ₘ-(Z)_{g}-CONR⁷SO₂NR¹³R¹³, —(CH₂)ₙ—SO₂NR¹³R¹³, -Het-(CH₂)ₘ—SO₂NR¹³R¹³, —(CH₂)ₙ-(Z)_{g}-SO₂NR¹³R¹³, -Het-(CH₂)ₘ-(Z)_{g}-SO₂NR¹³R¹³, —(CH₂)ₙ—NR¹⁰—(CH₂)ₘ(CHOR⁸)ₙ—SO₂NR¹³R¹³, -Het-(CH₂)ₘ—NR¹⁰—(CH₂)ₘ(CHOR⁸)ₙ—SO₂NR¹³R¹³, —(CH₂)ₙ—(CHOR⁸)ₘ—SO₂NR¹³R¹³, -Het-(CH₂)ₘ—(CHOR⁸)ₘ—SO₂NR¹³R¹³, —(CH₂)ₙ—(CHOR⁸)ₘ-(Z)_{g}-SO₂NR¹³R¹³, -Het-(CH₂)ₙ—(CHOR⁸)ₘ-(Z)_{g}-SO₂NR¹³R¹³, —(CH₂)ₙ-(Z)_{g}-(CH₂)ₘSO₂NR¹³R¹³, -Het-(CH₂)ₙ-(Z)_{g}-(CH₂)ₘSO₂NR¹³R¹³, —(CH₂)ₙ-(Z)_{g}-(CHOR⁸)ₘ-(Z)_{g}-SO₂NR¹³R¹³, -Het-(CH₂)ₙ-(Z)_{g}-(CHOR⁸)ₘ-(Z)_{g}-SO₂NR¹³R¹³, —(CH₂)ₙ—CONR¹³R¹³R¹³, -Het-(CH₂)ₘ—CONR¹³R¹³, —(CH₂)ₙ-(Z)_{g}-CONR¹³R¹³, -Het-(CH₂)ₘ-(Z)_{g}-CONR¹³R¹³, —(CH₂)ₙ—NR¹⁰—(CH₂)ₘ(CHOR⁸)ₙ—CONR¹³R¹³, -Het-(CH₂)ₘ—NR¹⁰—(CH₂)ₘ(CHOR⁸)ₙ—CONR¹³R¹³, —(CH₂)ₙ—(CHOR⁸)ₘ—CONR¹³R¹³, -Het-(CH₂)ₘ—(CHOR⁸)ₘ—CONR¹³R¹³, —(CH₂)ₙ—(CHOR⁸)ₘ-(Z)_{g}-CONR¹³R¹³, -Het-(CH₂)ₙ—(CHOR⁸)ₘ-(Z)_{g}-CONR¹³R¹³, —(CH₂)ₙ-(Z)_{g}-(CH₂)ₘCONR¹³R¹³, -Het-(CH₂)ₙ-(Z)_{g}-(CH₂)ₘCONR¹³R¹³, —(CH₂)ₙ-(Z)_{g}-(CHOR⁸)ₘ-(Z)_{g}-CONR¹³R¹³, -Het-(CH₂)ₙ-(Z)_{g}-(CHOR⁸)ₘ-(Z)_{g}-CONR¹³R¹³, —(CH₂)ₙ—CONR⁷COR¹³, -Het-(CH₂)ₘ—CONR⁷COR¹³, —(CH₂)ₙ-(Z)_{g}-CONR⁷COR¹³, -Het-(CH₂)ₘ-(Z)_{g}-CONR⁷COR¹³, —(CH₂)ₙ—NR¹⁰—(CH₂)ₘ(CHOR⁸)ₙ—CONR⁷COR¹³, -Het-(CH₂)ₘ-NR¹⁰—(CH₂)ₘ(CHOR⁸)ₙ—CONR⁷COR¹³, —(CH₂)ₙ—(CHOR⁸)ₘ—CONR⁷COR¹³, -Het-(CH₂)ₘ—(CHOR⁸)ₘ—CONR⁷COR¹³, —(CH₂)ₙ—(CHOR⁸)ₘ-(Z)_{g}-CONR⁷COR¹³ -Het-(CH₂)ₙ—(CHOR⁸)ₘ-(Z)_{g}-CONR⁷COR¹³, —(CH₂)ₙ-(Z)_{g}-(CH₂)ₘCONR⁷COR¹³, —(CH₂)ₙ-(Z)_{g}-(CH₂)ₘCONR⁷COR¹³, -Het-(CH₂)ₙ-(Z)_{g}-(CHOR⁸)ₘ-(Z)_{g}-CONR⁷COR¹³, —(CH₂)ₙ—CONR⁷CO₂R¹³, —(CH₂)ₙ-(Z)_{g}-CONR⁷CO₂R¹³, -Het-(CH₂)ₘ-(Z)_{g}-CONR⁷CO₂R¹³, —(CH₂)ₙ—NR¹⁰—(CH₂)ₘ(CHOR⁸)ₙ—CONR⁷CO₂R¹³, -Het-(CH₂)ₘ—NR¹⁰—(CH₂)ₘ(CHOR⁸)ₙ—CO NR⁷CO₂R¹³, —(CH₂)ₙ—(CHOR⁸)ₘ—CONR⁷CO₂R¹³, -Het-(CH₂)ₘ—(CHOR⁸)ₘ—CONR⁷CO₂R¹³, —(CH₂)ₙ—(CHOR⁸)ₘ-(Z)_{g}-CONR⁷CO₂R¹³, -Het-(CH₂)ₙ—(CHOR⁸)ₘ-(Z)_{g}-CONR⁷CO₂R¹³, —(CH₂)ₙ-(Z)_{g}-(CH₂)ₘCONR⁷CO₂R¹³, -Het-(CH₂)ₙ-(Z)_{g}-(CH₂)ₘCONR⁷CO₂R¹³, —(CH₂)ₙ-(Z)_{g}-(CHOR⁸)ₘ-(Z)_{g}-CONR⁷CO₂R¹³, -Het-(CH₂)ₘ-(Z)_{g}-(CHOR⁸)ₘ-(Z)_{g}-CONR⁷CO₂R¹³, —(CH₂)ₙ—NH—C(═NR¹³)—NR¹³R¹³, -Het-(CH₂)ₘ—NH—C(═NR¹³)—NR¹³R¹³, —(CH₂)ₙ-(Z)_{g}-NH—C(═NR¹³)—NR¹³R¹³, -Het-(CH₂)ₘ-(Z)_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, -Het-(CH₂)ₘ—NR¹⁰—(CH₂)ₘ(CHOR⁸)ₙ—NH—C(═NR¹³)—NR¹³R¹³, —(CH₂)ₙ—(CHOR⁸)ₘ—NH—C(═NR¹³)—NR¹³R¹³, -Het-(CH₂)ₘ—(CHOR⁸)ₘ—NH—C(═NR¹³)—NR¹³R¹³, —(CH₂)ₙ—(CHOR⁸)ₘ-(Z)_{g}-NH—C(═NR¹³)—NR¹³R¹³, -Het-(CH₂)ₙ—(CHOR⁸)ₘ-(Z)_{g}-NH—C(═NR¹³)—NR¹³R¹³, (CH₂)ₙ-(Z)_{g}-(CH₂)ₘNH—C(═NR¹³)—NR¹³R¹³, -Het-(CH₂)ₙ-(Z)_{g}-(CH₂)ₘNH—C(═NR¹³)—NR¹³R¹³, —(CH₂)ₙ-(Z)_{g}-(CHOR⁸)ₘ-(Z_{g}-NH—C(═NR¹³)—NR¹³R¹³, -Het-(CH₂)ₙ-(Z)_{g}-(CHOR⁸)ₘ-(Z)_{g}-NH—C(═NR¹³)—NR¹³R¹³, —(CH₂)ₙ—C(═NR¹³)—NR¹³R¹³, Het-(CH₂)ₘ—C(═NH)—NR¹³R¹³, —(CH₂)ₙ-(Z)_{g}-C(═NH)—NR¹³R¹³, Het-(CH₂)ₘ-(Z)_{g}-C(═NH)—NR¹³R¹³, —(CH₂)ₙ—NR¹⁰—(CH₂)ₘ(CHOR⁸)ₙ—C(═NR¹³)—NR¹³R¹³, Het-(CH₂)ₘ—NR¹⁰—(CH₂)ₘ(CHOR⁸)ₙ—C(═NR¹³)—NR¹³R¹³, —(CH₂)ₙ—(CHOR⁸)ₘ—C(═NR¹³)—NR¹³R¹³, -Het-(CH₂)ₘ—(CHOR⁸)ₘ—C(═NR¹³)—NR¹³R¹³, —(CH₂)ₙ—(CHOR⁸)ₙ-(Z)_{g}-C(═NR¹³)—NR¹³R¹³, -Het-(CH₂)ₘ—(CHOR⁸)ₘ-(Z)_{g}-C(═NR¹³)—NR¹³R¹³, —(CH₂)ₙ-(Z)_{g}-(CH₂)ₘ—C(═NHC(═NR¹³)—NR¹³R¹³, Het—(CH₂)ₙ-(Z)_{g}-(CH₂)ₘ—C(═N R¹³)—NR¹³R¹³, —(CH₂)ₙ-(Z)_{g}-(CHOR⁸)ₘ-(Z)_{g}-C(═NR¹³)—NR¹³R¹³, -Het-(CH₂)ₙ-(Z)_{g}-(CHOR⁸)ₘ-(Z)_{g}-C(═NR¹³)—NR¹³R¹³, —(CH₂)ₙ—NR¹²R¹², —O—(CH₂)ₘ—NR¹²R¹², —O—(CH₂)ₙ—NR¹²R¹², —O—(CH₂)ₘ(Z)_{g}R¹², —(CH₂)ₙNR¹¹R¹¹, —O—(CH₂)ₘNR¹¹R₁₁, —(CH₂)ₙ—N⊕—(R¹¹)₃, —O—(CH₂)ₘ—N⊖—(R¹¹)₃, —(CH₂)ₙ-(Z)_{g}-(CH₂)ₘ—NR¹⁰R¹⁰, —O—(CH₂)ₘ-(Z)_{g}-(CH₂)ₘ—NR¹⁰R¹⁰, —(CH₂CH₂O)ₘ—CH₂CH₂NR¹²R¹², —O—(CH₂CH₂O)ₘ—CH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰-(Z)_{g}-R¹⁰, —(CH₂)ₙ—(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g}-R¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂O)ₘ—R⁸, -(Het)-(CH₂CH₂O)ₘ—CH₂CH₂NR⁷R¹⁰, -(Het)-(CH₂)ₘ—C(═O)NR⁷R¹⁰, -(Het)-(CH₂)ₘ-(Z)_{g}-R⁷, -(Het)-(CH₂)ₘ—NR¹⁰—CH₂(CHOR⁸)(CHOR⁸)ₙ—CH₂OR⁸, -(Het)-(CH₂)ₘ—CO₂R⁷, -(Het)-(CH₂)ₘ—NR¹²R¹², -(Het)-(CH₂)ₙ—NR¹²R¹², -(Het)-(CH₂)ₙ-(Z)_{g}R¹², -(Het)-(CH₂)ₘNR¹¹R¹¹, -(Het)-(CH₂)ₘ—N⊕—(R¹¹)₃, -(Het)-(CH₂)ₘ-(Z)_{g}-(CH₂)ₘ—NR¹⁰R¹⁰, -(Het)-(CH₂CH₂O)ₘ—CH₂CH₂NR¹²R¹², -(Het)-(CH₂)ₘ—(C═O)NR¹²R¹², -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, Link-(CH₂)ₙ-CAP, Link-(CH₂)ₙ(CHOR⁸)(CHOR⁸)ₙ-CAP, Link-(CH₂CH₂O)ₘ—CH₂-CAP, Link-(CH₂CH₂O)ₘ—CH₂CH₂-CAP, Link-(CH₂)ₙ-(Z)_{g}-CAP, Link-(CH₂)ₙ(Z)_{g}-(CH₂)ₘ-CAP, Link-(CH₂)ₙ—NR¹³—CH₂(CHOR⁸)(CHOR⁸)ₙ-CAP, Link-(CH₂)ₙ—(CHOR⁸)ₘCH₂—NR¹³-(Z)_{g}-CAP, Link-(CH₂)ₙNR¹³—(CH₂)ₘ(CHOR⁸)ₙCH₂NR¹³-(Z)_{g}-CAP, -Link-(CH₂)ₘ-(Z)_{g}-(CH₂)ₘ-CAP, Link-NH—C(═O)—NH—(CH₂)ₘ-CAP, Link-(CH₂)ₘ—C(═O)NR¹³—(CH₂)ₘ—C(═O)NR¹⁰R¹⁰, Link-(CH₂)ₘ—C(═O)NR¹³—(CH₂)ₘ-CAP, Link-(CH₂)ₘ—C(═O)NR¹¹R¹¹, Link (CH₂)ₘ—C(═O)NR₁₂R₁₂, Link-(CH₂)ₙ-(Z)_{g}-(CH₂)ₘ-(Z)_{g}-CAP, Link-(Z)_{g}-(CH₂)ₘ-Het-(CH₂)ₘ-CAP, Link-(CH₂)ₙ—CR¹¹R¹¹-CAP, Link-(CH₂)ₙ(CHOR⁸)(CHOR⁸)ₙ—CR¹¹R¹¹-CAP, Link -(CH₂CH₂O)ₘ—CH₂—CR¹¹R¹¹-CAP, Link-(CH₂CH₂O)ₘ—CH₂CH₂—CR¹¹R¹¹-CAP, Link-(CH₂)ₙ-(Z)_{g}-CR¹¹R¹¹-CAP, Link-(CH₂)ₙ(Z)_{g}-(CH₂)ₘ—CR¹¹R¹¹-CAP, Link -(CH₂)ₙ—NR¹³—CH₂(CHOR⁸)(CHOR⁸)ₙ—CR¹¹R¹¹-CAP, Link-(CH₂)ₙ—(CHOR⁸)ₘCH₂—NR¹³-(Z)_{g}-CR¹¹R¹¹-CAP, Link-(CH₂)ₙNR¹³—(CH₂)ₘ(CHOR⁸)ₙCH₂NR¹³-(Z)_{g}-CR¹¹R¹¹-CAP, Link-(CH₂)ₘ-(Z)_{g}-(CH₂)ₘ—CR¹¹R¹¹-CAP, Link NH—C(═O)—NH—(CH₂)ₘ—CR¹¹R¹¹-CAP, Link (CH₂)ₘ—C(═O)NR¹³—(CH₂)ₘ—CR¹¹R¹¹-CAP, Link-(CH₂)ₙ-(Z)_{g}-(CH₂)ₘ-(Z)_{g}-CR¹¹R¹¹-CAP, or Link-(Z)_{g}-(CH₂)ₘ-Het-(CH₂)ₘ—CR¹¹R¹¹-CAP;

each R⁶ is, independently, R⁵, —R⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R^{1O}, —(CH₂)ₙ—C(═O)NR⁷R¹⁰, —O—(CH₂)ₙ—C(═O)NR⁷R¹⁰, —(CH₂)ₙ-(Z)_{g}-R⁷, —O—(CH₂)ₘ-(Z)_{g}-R⁷, —(CH₂)ₘ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

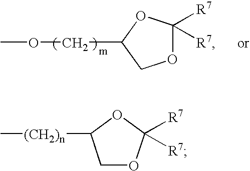

wherein when two R⁶ are —OR¹¹ and are located adjacent to each other on the aromatic carbocycle or aromatic heterocycle, the two Or¹¹ may form a methylenedioxy group;

each R⁷ is, independently, hydrogen, lower alkyl, phenyl, substituted phenyl or —CH₂(CHOR⁸)ₘ—CH₂OR⁸;

each R⁸ is, independently, hydrogen, lower alkyl, —C(═O)—R¹¹, glucuronide, 2-tetrahydropyranyl, or

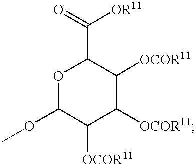

each R⁹ is, independently, —CO₂R⁷, —CON(R⁷)₂, —SO₂CH₃, —C(═O)R⁷, —CO₂R¹³, —CON(R¹³)₂, —SO₂CH₂R¹³, or —C(═O)R¹³;

each R¹⁰ is, independently, —H, —SO₂CH₃, —CO₂R⁷, —C(═O)NR⁷R⁹, —C(═O)R⁷, or —CH₂—(CHOH)ₙ—CH₂OH;

each Z is, independently, —(CHOH)—, —C(═O)—, —(CHNR⁷R¹⁰)—, —(C═NR¹⁰)—, —NR¹⁰—, —(CH₂)ₙ—, —(CHNR¹³R¹³)—, —(C═NR¹³)—, or —NR¹³—;

each R¹¹ is, independently, hydrogen, lower alkyl, phenyl lower alkyl or substituted phenyl lower alkyl;

each R¹² is, independently, —SO₂CH₃, —CO₂R⁷, —C(═O)NR⁷R⁹, —C(═O)R⁷, —CH₂(CHOH)ₙ—CH₂OH, —CO₂R¹³, —C(═O)NR¹³R¹³, or —C(═O)R¹³;

each R¹³ is, independently, R⁷, R¹⁰, —(CH₂)ₘ—NR⁷R¹⁰, —(CH₂)ₘ—NR⁷R⁷, —(CH₂)ₘ—NR¹¹R¹¹, —(CH₂)ₘ—(NR¹¹R¹¹R¹¹)⁺, —(CH₂)ₘ, —(CHOR⁸)ₘ—(CH₂)ₘNR¹¹R¹¹, —(CH₂)ₘ—(CHOR⁸)ₘ—(CH₂)ₘNR⁷R¹⁰, —(CH₂)ₘ—NR¹⁰R¹⁰, —(CH₂)ₘ—(CHOR⁸)ₘ—(CH₂)ₘ—(NR¹¹R¹¹R¹¹)⁺, —(CH₂)ₘ—(CHOR⁸)ₘ—(CH₂)ₘNR⁷R⁷,

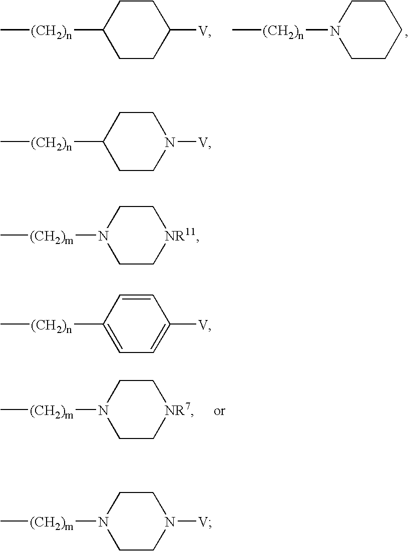

with the proviso that in the moiety —NR¹³R¹³, the two R¹³ along with the nitrogen to which they are attached may, optionally, form a ring selected from:

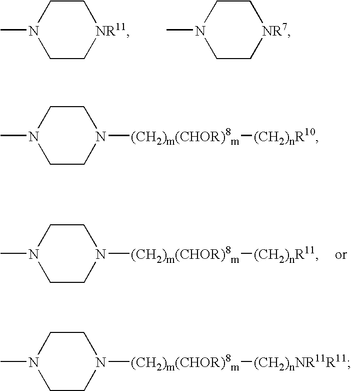

each V is, independently, —(CH₂)ₘ—NR⁷R¹⁰, —(CH₂)ₘ—NR⁷R⁷, —(CH₂)ₘ—(NR^{11 R}¹¹R¹¹)⁺, —(CH₂)ₙ—(CHOR⁸)ₘ—(CH₂)ₘNR⁷R¹⁰, —(CH₂)ₙ—NR¹⁰R¹⁰—(CH₂)ₙ—(CHOR⁸)ₘ—(CH₂)ₘNR⁷R⁷, —(CH₂)ₙ—(CHOR⁸)ₘ—(CH₂)ₘ—(NR¹¹R¹¹R¹¹)⁺ with the proviso that when V is attached directly to a nitrogen atom, then V can also be, independently, R⁷, R¹⁰, or (R¹¹)₂;

each R¹⁴ is, independently, H, R¹², —(CH₂)ₙ—SO₂CH₃, —(CH₂)ₙ—CO₂R¹³, —(CH₂)ₙ—C(═O)NR¹³R¹³, —(CH₂)ₙ—C(═O)R¹³, —(CH₂)ₙ—(CHOH)ₙ—CH₂OH, —NH—(CH₂)ₙ—SO₂CH₃, NH—(CH₂)ₙ—C(═O)R¹¹, NH—C(═O)—NH—C(═O)R¹¹, —C(═O)NR¹³R¹³, —OR¹¹, —NH—(CH₂)ₙ—R¹⁰, —Br, —Cl, —F, —I, SO₂NHR¹¹, —NHR¹³, —NH—C(═O)—NR¹³R¹³, —(CH₂)ₙ—NHR¹³, or —NH—(CH₂)ₙ—C(═O)—R¹³;

each g is, independently, an integer from 1 to 6;

each m is, independently, an integer from 1 to 7;

each n is, independently, an integer from 0 to 7;

each -Het- is, independently, —N(R⁷)—, —N(R¹⁰)—, —S—, —SO—, —SO₂—; —O—, —SO₂NH—, —NHSO₂—, —NR⁷CO—, —CONR⁷—, —N(R¹³)—, —SO₂NR¹³—, —NR¹³CO—, or —CONR¹³—;

each Link is, independently, —O—, —(CH₂)ₙ—, —O(CH₂)ₘ—, —NR¹³—C(═O)—NR³—, —NR¹³—C(═O)—(CH₂)ₘ—, —C(═O)NR¹³—(CH₂)ₘ, —(CH₂)ₙ-(Z)_{g}(CH₂)ₙ—, —S—, —SO—, —SO₂—, —SO₂NR⁷—, —SO₂NR¹⁰—, or -Het-;

each CAP is, independently, thiazolidinedione, oxazolidinedione, -heteroaryl-C(═O)N R¹³R¹³, heteroaryl-W, —CN, —O—C(═S)NR¹³R¹³, -(Z)_{g}R¹³, —CR¹⁰((Z)_{g}R¹³)((Z)_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, a cyclic sugar or oligosaccharide, a cyclic amino sugar, oligosaccharide, —CR¹⁰(—(CH₂)ₘ—R⁹)(—(CH₂)ₘ—R⁹), —N(—(CH₂)ₘ—R⁹), —(CH²)ₘ—R⁹), —NR¹³(—(CH₂)ₘ—CO₂R¹³),

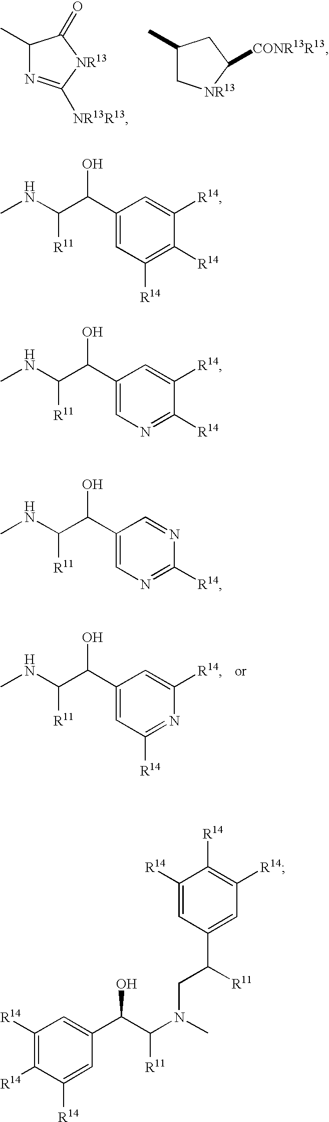

each Ar is, independently, phenyl, substituted phenyl, wherein the substituents of the substituted phenyl are 1-3 substituents independently selected from the group consisting of OH, OCH₃, NR¹³R¹³, Cl, F, and CH₃, or heteroaryl; and

each W is, independently, thiazolidinedione, oxazolidinedione, heteroaryl-C(═O)N R¹³R¹³, —CN, —O—C(═S)NR¹³R¹³, -(Z)_{g}R¹³, —CR¹⁰((Z)_{g}R¹³)( (Z)_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, a cyclic sugar or oligosaccharide, a cyclic amino sugar, oligosaccharide,

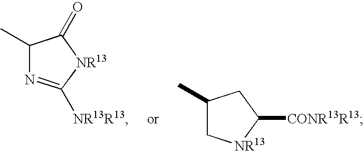

with the proviso that when any —CHOR⁸— or CH₂OR⁸ groups are located 1,2- or 1,3- with respect to each other, the R⁸ groups may, optionally, be taken together to form a cyclic mono- or di-substituted 1,3-dioxane or 1,3-dioxolane.

In the compounds represented by formula (I), X may be hydrogen, halogen, trifluoromethyl, lower alkyl, lower cycloalkyl, unsubstituted or substituted phenyl, lower alkyl-thio, phenyl-lower alkyl-thio, lower alkyl-sulfonyl, or phenyl-lower alkyl-sulfonyl. Halogen is preferred.

Examples of halogen include fluorine, chlorine, bromine, and iodine. Chlorine and bromine are the preferred halogens. Chlorine is particularly preferred. This description is applicable to the term “halogen” as used throughout the present disclosure.

As used herein, the term “lower alkyl” means an alkyl group having less than 8 carbon atoms. This range includes all specific values of carbon atoms and subranges there between, such as 1, 2, 3, 4, 5, 6, and 7 carbon atoms. The term “alkyl” embraces all types of such groups, e.g., linear, branched, and cyclic alkyl groups. This description is applicable to the term “lower alkyl” as used throughout the present disclosure. Examples of suitable lower alkyl groups include methyl, ethyl, propyl, cyclopropyl, butyl, isobutyl, etc.

Substituents for the phenyl group include halogens. Particularly preferred halogen substituents are chlorine and bromine.

Y may be hydrogen, hydroxyl, mercapto, lower alkoxy, lower alkyl-thio, halogen, lower alkyl, lower cycloalkyl, mononuclear aryl, or —N(R²)₂. The alkyl moiety of the lower alkoxy groups is the same as described above. Examples of mononuclear aryl include phenyl groups. The phenyl group may be unsubstituted or substituted as described above. The preferred identity of Y is —N(R²)₂. Particularly preferred are such compounds where each R² is hydrogen.

R¹ may be hydrogen or lower alkyl. Hydrogen is preferred for R¹.

Each R² may be, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-(Z)_{g}R⁷, —(CH₂)ₘ—NR¹⁰—CH₂(CHOR⁸)(CHOR⁸)ₙ—CH₂OR⁸, —(CH₂)ₙ—CO₂R⁷, or

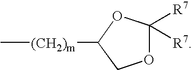

Hydrogen and lower alkyl, particularly C₁-C₃ alkyl, are preferred for R². Hydrogen is particularly preferred.

R³ and R⁴ may be, independently, hydrogen, lower alkyl, hydroxyl-lower alkyl, phenyl, (phenyl)-lower alkyl, (halophenyl)-lower alkyl, ((lower-alkyl)phenyl)-lower-alkyl), (lower-alkoxyphenyl)-lower alkyl, (naphthyl)-lower alkyl, (pyridyl)-lower alkyl or a group represented by —(C(R^{L})₂)ₒ-x-(C(R^{L})₂)ₚA¹ or —(C(R^{L})₂)ₒ-x-(C(R^{L})₂)ₚA², provided that at least one of R³ and R⁴ is a group represented by —(C(R^{L})₂)ₒ-x-(C(R^{L})₂)ₚA¹ or —(C(R^{L})₂)ₒ-x-(C(R^{L})₂)ₚA².

Preferred compounds are those where one of R³ and R⁴ is hydrogen and the other is represented by —(C(R^{L})₂)ₒ-x-(C(R^{L})₂)ₚA¹ or —(C(R^{L})₂)ₒ-x-(C(R^{L})₂)ₚA². In a particularly preferred aspect one of R³ and R⁴ is hydrogen and the other of R³ or R⁴ is represented by —(C(R^{L})₂)ₒ-x-(C(R^{L})₂)ₚA¹. In another particularly preferred aspect one of R³ and R⁴ is hydrogen and the other of R³ or R⁴ is represented by —(C(R^{L})₂)ₒ-x-(C(R^{L})₂)ₚA².

A moiety —(C(R^{L})₂)ₒ-x-(C(R^{L})₂)ₚ- defines an alkylene group bonded to the group A¹ or A₂. The variables o and p may each, independently, be an integer from 0 to 10, subject to the proviso that the sum of o and p in the chain is from 1 to 10. Thus, o and p may each be 0, 1, 2, 3, 4, 5, 6, 7, 8, 9, or 10. Preferably, the sum of o and p is from 2 to 6. In a particularly preferred embodiment, the sum of o and p is 4.

The linking group in the alkylene chain, x, may be, independently, O, NR¹⁰, C(═O), CHOH, C(═N—R¹⁰), CHNR⁷R¹⁰, or a single bond;

Therefore, when x is a single bond, the alkylene chain bonded to the ring is represented by the formula —(C(R^{L})₂)ₒ₊ₚ—, in which the sum o+p is from 1 to 10.

Each R^{L} may be, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

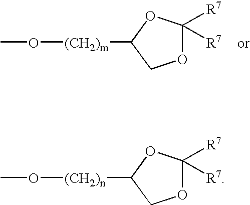

The term —O-glucuronide, unless otherwise specified, means a group represented by

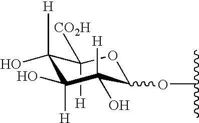

wherein the [symbol] O means the glycosidic linkage can be above or below the plane of the ring.

The term —O-glucose, unless otherwise specified, means a group represented by

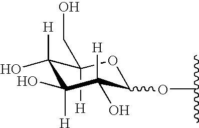

wherein the [symbol] O means the glycosidic linkage can be above or below the plane of the ring.

The preferred R^{L} groups include —H, —OH, —N(R⁷)₂, especially where each R⁷ is hydrogen.

In the alkylene chain in —(C(R^{L})₂)ₒ-x-(C(R^{L})₂)ₚA¹ or —(C(R^{L})₂)ₒ-x-(C(R^{L})₂)ₚA², it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, wherein the other R^{L} groups in the chain are hydrogens. Even more preferably, only one R^{L} group in an alkylene chain is other than hydrogen, wherein the other R^{L} groups in the chain are hydrogens. In these embodiments, it is preferable that x is a single bond.

In another particular embodiment of the invention, all of the R^{L} groups in the alkylene chain are hydrogen. In these embodiments, the alkylene chain is represented by the formula —(CH₂)ₒ-x-(CH₂)ₚ—.

A¹ is a C₆-C₁₅-membered aromatic carbocycle substituted with at least one R⁵ and the remaining substituents are R⁶. The term aromatic is well known term of chemical art and designates conjugated systems of 4n′+2 electrons that are within a ring system, that is with 6, 10, 14, etc. π-electrons wherein, according to the rule of Huckel, n′ is 1, 2, 3, etc. The 4n′+2 electrons may be in any size ring including those with partial saturation so long as the electrons are conjugated. For instance, but not by way of limitation, 5H-cyclohepta-1,3,5-triene, benzene, naphthalene, 1,2,3,4-tetrahydronaphthalene etc. would all be considered aromatic.

The C₆-C₁₅ aromatic carbocycle may be monocyclic, bicyclic, or tricyclic and may include partially saturated rings. Non-limiting examples of these aromatic carbocycles comprise benzene, 5H-cyclohepta-1,3,5-triene, naphthalene, phenanthrene, azulene, anthracene, 1,2,3,4-tetrahydronapthalene, 1,2-dihydronapthalene, indene, 5H-dibenzo[a,d]cycloheptene, etc.

The C₆-C ₁₅ aromatic carbocycle may be attached to the —(C(R^{L})₂)ₒ-x-(C(R^{L})₂)ₚ-moiety through any ring carbon atom as appropriate, unless otherwise specified. Therefore, when partially saturated bicyclic aromatic is 1,2-dihydronapthalene, it may be 1,2-dihydronapthalen-1-yl, 1,2-dihydronapthalen-3-yl, 1,2-dihydronapthalen-5-yl, etc. In a preferred embodiment A¹ is phenyl, indenyl, napthalenyl, 1,2-dihydronapthalenyl, 1,2,3,4-tetrahydronapthalenyl, anthracenyl, fluorenyl, phenanthrenyl, azulenyl, cyclohepta-1,3,5-trienyl or 5H-dibenzo[a,d]cycloheptenyl. In another preferred embodiment, A¹ is phenyl. In another preferred embodiment, A¹ is napthalen-1-yl. In another preferred embodiment, A¹ is napthalen-2-yl.

In another preferred embodiment, A¹ is

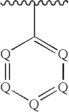

wherein each Q is, independently, C—H, C—R⁵, or C—R⁶, with the proviso that at least one Q is C—R⁵. Therefore, Q may be 1, 2, 3, 4, or 5 C—H. In a particularly preferred embodiment, each R⁶ is H.

In another preferred embodiment, A¹ is

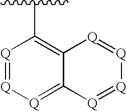

wherein each Q is, independently, C—H, C—R⁵, or C—R⁶, with the proviso that at least one Q is C—R⁵. Therefore, Q may be 1, 2, 3, 4, 5, or 6 C—H. Therefore, Q may be 1, 2, 3, 4, 5, or 6 C—R⁶. In a particularly preferred embodiment, each R⁶ is H.

In another preferred embodiment, A¹ is

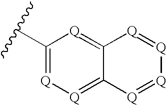

wherein each Q is, independently, C—H, C—R⁵, C—R⁶, with the proviso that at least one Q is C—R⁵. Therefore, Q may be 1, 2, 3, 4, 5, or 6 C—H. Therefore, Q may be 1, 2, 3, 4, 5, or 6 C—R⁶. In a particularly preferred embodiment, each R⁶ is H.

In a particularly preferred embodiment, A¹ is

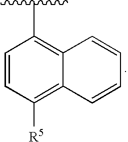

In another particularly preferred embodiment, A¹ is

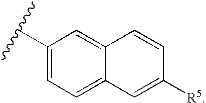

In another particularly preferred embodiment, A¹ is

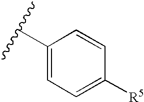

A² is a six to fifteen-membered aromatic heterocycle substituted with at least one R⁵ and the remaining substituents are R⁶ wherein the aromatic heterocycle comprises 1-4 heteroatoms selected from the group consisting of O, N, and S.

The six to fifteen-membered aromatic heterocycle may be monocyclic, bicyclic, or tricyclic and may include partially saturated rings. Non limiting examples of these aromatic heterocycles include pyridyl, 1H-azepine, benzo[b]furan, benzo[b]thiophene, isobenzofuran, isobenzothiophene, 2,3-dihydrobenzo[b]furan, benzo[b]thiophene, 2,3-diydrobenzo[b]thiophene, indolizine, indole, isoindole benzoxazole, benzimidazole, indazole, benzisoxazole, benzisothizole, benzopyrazole, benzoxadiazole, benzothiadiazole, benzotriazole, purine, quinoline, 1,2,3,4-tetrahydroquinoline, 3,4-dihydro-2H-chromene, 3,4-dihydro-2H-thiochromene, isoquinoline, cinnoline, quinolizine, phthalazine, quinoxaline, quinazoline, naphthiridine, pteridine, benzopyrane, pyrrolopyridine, pyrrolopyrazine, imidazopyrdine, pyrrolopyrazine, thienopyrazine, furopyrazine, isothiazolopyrazine, thiazolopyrazine, isoxazolopyrazine, oxazolopyrazine, pyrazolopyrazine, imidazopyrazine, pyrrolopyrimidine, thienopyrimidine, furopyrimidine, isothiazolopyrimidine, thiazolopyrimidine, isoxazolopyrimidine, oxazolopyrimidine, pyrazolopyrimidine, imidazopyrimidine, pyrrolopyridazine, thienopyridazine, furopyridazine, isothiazolopyridazine, thiazolopyridazine, oxazolopyridazine, thiadiazolopyrazine, oxadiazolopyrimidine, thiadiazolopyrimidine, oxadiazolopyridazine, thiazolopyridazine, imidazooxazole, imidazothiazole, imidazoimidazole, isoxazolotriazine, isothiazolotriazine, oxazolotriazine, thiazolotriazine, carbazole, acridine, phenazine, phenothiazine, phenooxazine, and 5H-dibenz[b,f]azepine, 10,11-dihydro-5H-dibenz[b,f]azepine, etc.

The six to fifteen-membered aromatic heterocycle may be attached to the —(C(R^{L})₂)ₒ-x-(C(R^{L})₂)ₚ-moiety through any ring carbon atom or ring nitrogen atom so long as a quanternary nitrogen atom is not formed by the attachment. Therefore, when partially saturated aromatic heterocycle is 1H-azepine, it may be 1H-azepin-1-yl, 1H-azepin-2-yl, 1H-azepin-3-yl, etc. Preferred aromatic heterocycles are pyridyl, indolizinyl, indolyl, isoindolyl, indolinyl, benzo[b]furanyl, 2,3-dihydrobenzo[b]furanyl, benzo[b]thiophenyl, 2,3-diydrobenzo[b]thiophenyl, indazolyl, benzimidazolyl, benzthiazolyl, purinyl, quinolinyl, 1,2,3,4-tetrahydroquinolinyl, 3,4-dihydro-2H-chromenyl, 3,4-dihydro-2H-thiochromenyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, dibenzofuranyl, dibenzothiophenyl, 1H-azepinyl, 5H-dibenz[b,f]azepinyl, are 10,11-dihydro-5H-dibenz[b,f]azepinyl.

In another preferred embodiment, A² is

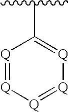

wherein each Q is, independently, C—H, C—R⁵, C—R⁶, or a nitrogen atom, with the proviso that at least one Q is nitrogen and one Q is C—R⁵, and at most three Q in a ring are nitrogen atoms. Therefore, in any one ring, each Q may be 1, 2, or 3 nitrogen atoms. In a preferred embodiment, only one Q in each ring is nitrogen. In another preferred embodiment, only a single Q is nitrogen. Optionally, 1, 2, 3, or 4 Q may be C—R⁶. Optionally, Q may be 1, 2, 3, or 4 C—H. In a particularly preferred embodiment, each R⁶ is H.

In another preferred embodiment, A² is

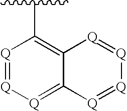

wherein each Q is, independently, C—H, C—R⁵, C—R⁶, or a nitrogen atom, with the proviso that at least one Q is nitrogen and one Q is C—R⁵, and at most three Q in a ring are nitrogen atoms. Therefore, in any one ring, each Q may be 1, 2, or 3 nitrogen atoms. In a preferred embodiment, only one Q in each ring is nitrogen. In another preferred embodiment, only a single Q is nitrogen. Optionally, 1, 2, 3, 4, or 5 Q may be C—R⁶. Optionally, Q may be 1, 2, 3, 4, or 5 C—H. In a particularly preferred embodiment, each R⁶ is H.

In another preferred embodiment, A² is

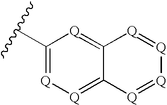

wherein each Q is, independently, C—H, C—R⁵, C—R⁶, or a nitrogen atom, with the proviso that at least one Q is nitrogen and one Q is C—R⁵, and at most three Q in a ring are nitrogen atoms. Therefore, in any one ring, each Q may be 1, 2, or 3 nitrogen atoms. In a preferred embodiment, only one Q in each ring is nitrogen. In another preferred embodiment, only a single Q is nitrogen. Optionally, Q may be 1, 2, 3, 4, or 5 C—H. Optionally, 1, 2, 3, 4, or 5 Q may be C—R⁶. In a particularly preferred embodiment, each R⁶ is H.

In a preferred embodiment R⁵ is one of the following: —(CH₂)ₘ—OR⁸, —(CH₂)₄—OH, —O—(CH₂)ₘ—OR⁸, —O—(CH₂)₄—OH, —(CH₂)ₙ—NR⁷R¹⁰, —NHSO₂CH₃, —CH₂NH(C═O)—(OCH₃)₃, —NH(C═O)CH₃, —CH₂NH₂, —NH—CO₂C₂H₅, —CH₂NH(C═O)CH₃, —CH₂NHCO₂CH₃, —CH₂NHSO₂CH₃, —(CH₂)₄—NH(C═O)O(CH₃)₃, —(CH₂)₄—NH₂, —(CH₂)₃—NH(C═O)O(CH₃)₃, —(CH₂)₃—NH₂, —O—(CH₂)ₘ—NR⁷R¹⁰, —OCH₂CH₂NHCO₂(CH₃)₃, —OCH₂CH₂NHCO₂C₂H₅, —O—(CH₂)₃—NH—CO₂—(CH₃)₃, —O(CH₂)₃—NH₂, —OCH₂CH₂NHSO₂CH₃, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —OCH₂CHOHCH₂O-glucuronide, —OCH₂CH₂CHOHCH₂OH, —OCH₂-(α—CHOH)₂CH₂OH, —OCH₂—(CHOH)₂CH₂OH, —(CH₂CH₂O)ₘ—R⁸, —O—(CH₂CH₂O)ₘ—R⁸, (CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —C(═O)NH₂, —O—(CH₂)ₘ—C(═O)NR⁷R¹⁰, —O—CH₂—(C═O)NHCH₂CHOH, —O—CH₂—(C═O)NHCH₂CHOHCH₂OH, —O—CH₂(C═O)NHCH₂(CHOH)₂CH₂OH, —O—CH₂C(C═O)NHSO₂CH₃, —O—CH₂(C═O)NHCO₂CH₃, —O—CH₂—C(C═O)NH—C(C═O)NH₂, —O—CH₂—(C═O)NH—(C═O)CH₃, —(CH₂)ₙ-(Z)_{g}-R⁷, —(CH₂)ₙ—(C═N)—NH₂, —(C═NH)NH₂, —(CH₂)ₙ—NH—C(═NH)—NH₂, —(CH₂)₃—NH—C(═NH)—NH₂, —CH₂NH—C(═NH)—NH₂, —(CH₂)ₙ—CONHCH₂(CHOH)ₙ—CH₂OH, —NH—C(═O)—CH₂—(CHOH)ₙCH₂OH, —NH—(C═O)—NH—CH₂(CHOH)₂CHOH, —NHC(C═O)NHCH₂CH₂OH, —O—(CH₂)ₘ-(Z)_{g}-R⁷, —O—(CH₂)ₘ—NH—C(═NH)—N(R⁷)₂, —O(CH₂)₃—NH—C(═NH)—NH₂, —O—(CH₂)ₘ—CHNH₂—CO₂NR⁷R¹⁰, —OCH₂—CHNH₂—CO₂NH₂, —O—(CH₂)ₘ—CHNH₂—CO₂NR⁷R¹⁰ (anomeric center is the (R) enantiomer), —O—(CH₂)ₘ—CHNH₂—CO₂NR⁷R¹⁰ (anomeric center is the (S) enantiomer), —OCH₂CHOH—CH₂NHCO₂(CH₃)₃, —(CH₂)ₙ—NR¹⁰—CH₂(CHOR⁸)(CHOR⁸)ₙ—CH₂OR⁸, —NHCH₂(CHOH)₂CH₂OH, —O—(CH₂)ₘ—NR¹⁰—CH₂(CHOR⁸)(CHOR⁸)ₙ—CH₂OR⁸, —O—(CH₂)ₘ—CO₂R⁷, —OCH₂CH₂CO₂(CH₃)₃, —OCH₂CO₂H, —OCH₂CO₂C₂H₅, —O—(CH₂)ₘ-Boc, —(CH₂)ₘ-Boc, —O—(CH₂)ₘ—NH—C(═NH)—N(R⁷)₂, —(CH₂)ₙ—NH—C(═NH)—N(R⁷)₂, —(CH₂)ₘ—NH—C(═O)—OR⁷, —O(CH₂)ₘ—NH—C(═O)—OR⁷, —(CH₂)ₙ—NH—C(═O)—R¹¹, —O—(CH₂)ₘ—NH—C(═O)—R¹¹, —O—(CH₂)ₘ—C(═O)N(R⁷)₂, —(CH₂)ₘ—CHOH—CH₂—NHBoc, —O(CH₂)ₘ—CHOH—CH₂—NHBoc, —(CH₂)ₘ—NHC(O)OR⁷, —O—(CH₂)ₘ—NHC(O)OR⁷, —O—(CH₂)ₘ—C(═NH)—N(R⁷)₂, or —(CH₂)ₙ—C(═NH)—N(R⁷)₂.

In another embodiment, R⁵ is selected from the group consisting of —O—(CH₂)₃—OH, —NH₂, —O—CH₂—(CHOH)₂—CH₂OH —O—CH₂—CHOH—CH₂OH, —O—CH₂CH₂—O-tetrahydropyran-2-yl, —O—CH₂CHOH—CH₂—O-glucuronide, —O—CH₂CH₂OH, —O—(CH₂CH₂O)₄—CH₃, —O—CH₂CH₂OCH₃, —O—CH₂—(CHOC(═O)CH₃)—CH₂—OC(═O)CH₃, —O—(CH₂CH₂O)₂—CH₃, —OCH₂—CHOH—CHOH—CH₂OH, —CH₂OH, —CO₂CH₃,

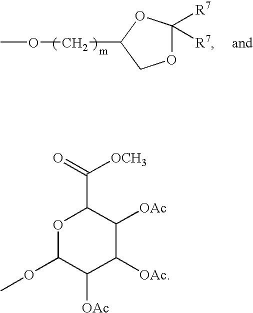

In another embodiment, R⁵ is selected from the group consisting of —O—(CH₂)₃—OH, —NH₂, —O—CH₂—(CHOH)₂—CH₂OH, —O—CH₂—CHOH—CH₂OH, —O—CH₂CH₂—O-tetrahydropyran-2-yl, —O—CH₂CHOH—CH₂—O-glucuronide, —O—CH₂CH₂OH, —O—(CH₂CH₂O)₄—CH₃, —O—CH₂CH₂OCH₃, —O—CH₂—(CHOC(═O)CH₃)—CH₂—OC(═O)CH₃, —O—(CH₂CH₂O)₂—CH₃, —OCH₂—CHOH—CHOH—CH₂OH, —CH₂OH, —CO₂CH₃, —SO₃H, —O-glucuronide,

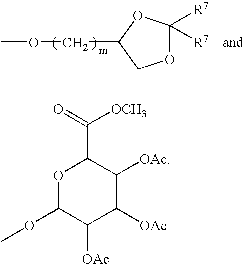

In a preferred embodiment, each —(CH₂)ₙ-(Z)_{g}-R⁷ falls within the scope of the structures described above and is, independently,

—(CH₂)ₙ—(C═N)—NH₂,

—(CH₂)ₙ—NH—C(═NH)NH₂,

—(CH₂)ₙ—CONHCH₂(CHOH)ₙ—CH₂OH, or

—NH—C(═O)—CH₂—(CHOH)ₙCH₂OH.

In another a preferred embodiment, each —O—(CH₂)ₘ-(Z)_{g}-R⁷ falls within the scope of the structures described above and is, independently,

—O—(CH₂)ₘ—NH—C(═NH)—N(R⁷)₂, or

—O—(CH₂)ₘ—CHNH₂—CO₂NR⁷R¹⁰.

In another preferred embodiment, R⁵ may be one of the following: —O—CH₂CHOHCH₂O-glucuronide, —OCH₂CHOHCH₃, —OCH₂CH₂NH₂, —OCH₂CH₂NHCO(CH₃)₃, —CH₂CH₂OH, —OCH₂CH₂OH, —O—(CH₂)ₘ-Boc, —(CH₂)ₘ-Boc, —OCH₂CH₂OH, —OCH₂CO₂H, —O—(CH₂)ₘ—NH—C(═NH)—N(R⁷)₂, —(CH₂)ₙ—NH—C(═NH)—N(R⁷)₂, —NHCH₂(CHOH)₂—CH₂OH, —OCH₂CO₂Et, —NHSO₂CH₃, —(CH₂)ₘ—NH—C(═O)—OR⁷, —O—(CH₂)ₘ—NH—C(═O)OR⁷, —(CH₂)ₙ—NH—C(═O)—R¹¹, —O—(CH₂)ₘ—NH—C(═O)—R¹¹, —O—CH₂C(═O)NH₂, —CH₂NH₂, —NHCO₂Et, —OCH₂CH₂CH₂CH₂OH, —CH₂NHSO₂CH₃, —OCH₂CH₂CHOHCH₂OH, —OCH₂CH₂NHCO₂Et, —NH—C(═NH2)—NH₂, —OCH₂—(α—CHOH)₂—CH₂OH, —OCH₂CHOHCH₂NH₂, —(CH₂)ₘ—CHOH—CH₂—NHBoc, —O—(CH₂)ₘ—CHOH—CH₂—NHBoc, —(CH₂)ₘ—NHC(O)OR⁷, —O—(CH₂)ₘ—NHC(O)OR⁷, —OCH₂CH₂CH₂NH₂, —OCH₂CH₂NHCH₂(CHOH)₂CH₂OH, —OCH₂CH₂NH(CH₂[(CHOH)₂CH₂OH)]₂, —(CH₂)₄—NHBoc, —(CH₂)₄—NH₂, OCH₂CH₂NHSO₂CH₃, —O—(CH₂)ₘ—C(═NH)—N(R⁷)₂, —(CH₂)ₙ—C(═NH)—N(R⁷)₂, —(CH₂)₃—NH Boc, —(CH₂)₃NH₂, —O—(CH₂)ₘ—NH—NH—C(═NH)—N(R⁷)₂, —(CH₂)ₙ—NH—NH—C(═NH)—N(R⁷)₂, —(CH₂)ₙ—NH—NH—C(═NH)—N(R⁷)₂, or —O—CH₂—CHOH—CH₂—NH—C(═NH)—N(R⁷)₂.

In another preferred embodiment, R⁵ is —OH, —O—(CH₂)ₘ(Z)_{g}R¹², -Het-(CH₂)ₘ—NH—C(═NR¹³)—NR¹³R¹³, -Het-(CH₂)ₙ-(Z)_{g}-(CH₂)ₘNH—C(═NR¹³)—NR¹³R¹³, -Link-(CH₂)ₘ-(Z)_{g}-(CH₂)ₘ-CAP, Link-(CH₂)ₙ—CR¹¹R¹¹-CAP, -Het-(CH₂)ₘ—CONR¹³R¹³, —(CH₂)ₘ—NR¹²R¹² , —O—(CH₂)ₘNR¹¹R¹¹, —O—(CH₂)ₘ—N⊕—(R¹¹)₃, —(CH₂)ₙ-(Z)_{g}-(CH₂)ₘ—NR¹⁰OR¹⁰, -Het-(CH₂)ₘ-(Z)_{g}-NH—C(═NR¹³)—NR¹³R¹³, —O—(CH₂)ₘ(CHOR⁸)(CHOR⁸)ₙ—CH₂OR⁸, —O—(CH₂)ₘ—C(═O)NR⁷R¹⁰, —O—(CH₂)ₘ-(Z)_{g}R⁷, or —O—(CH₂)ₘ—NR¹⁰—CH₂(CHOR⁸)(CHOR⁸)ₙ—CH₂OR⁸.

In a particularly preferred embodiment, R⁵ is —O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

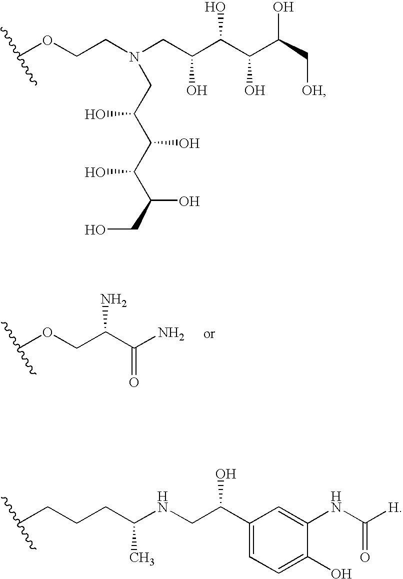

Selected substituents within the compounds of the invention are present to a recursive degree. In this context, “recursive substituent” means that a substituent may recite another instance of itself. Because of the recursive nature of such substituents, theoretically, a large number of compounds may be present in any given embodiment. For example, R⁹ contains a R¹³ substituent. R¹³ can contain an R¹⁰ substituent and R¹⁰ can contain a R⁹ substituent. One of ordinary skill in the art of medicinal chemistry understands that the total number of such substituents is reasonably limited by the desired properties of the compound intended. Such properties include, by way of example and not limitation, physical properties such as molecular weight, solubility or log P, application properties such as activity against the intended target, and practical properties such as ease of synthesis.

By way of example and not limitation, R⁹, R¹³ and R¹⁰ are recursive substituents in certain embodiments. Typically, each of these may independently occur 20, 19, 18, 17, 16, 15, 14, 13, 12, 11, 10, 9, 8, 7, 6, 5, 4, 3, 2, 1, or 0, times in a given embodiment. More typically, each of these may independently occur 12 or fewer times in a given embodiment. More typically yet, R⁹ will occur 0 to 8 times in a given embodiment, R¹³ will occur 0 to 6 times in a given embodiment and R¹⁰ will occur 0 to 6 times in a given embodiment. Even more typically yet, R⁹ will occur 0 to 6 times in a given embodiment, R¹³ will occur 0 to 4 times in a given embodiment and R¹⁰ will occur 0 to 4 times in a given embodiment.

Recursive substituents are an intended aspect of the invention. One of ordinary skill in the art of medicinal chemistry understands the versatility of such substituents. To the degree that recursive substituents are present in an embodiment of the invention, the total number will be determined as set forth above.

Each -Het- is, independently, —N(R⁷)—, —N(R¹⁰)—, —S—, —SO—, —SO₂—; —O—, —SO₂NH—, —NHSO₂—, —NR⁷CO—, —CONR⁷—, —N(R¹³)—, —SO₂NR¹³—, —NR¹³CO—, or —CONR¹³—. In a preferred embodiment, -Het- is —O—, —N(R⁷)—, or —N(R¹⁰)—. Most preferably, -Het- is —O—.

Each -Link- is, independently, —O—, —(CH₂)ₙ—, —O(CH₂)ₘ—, —NR¹³—C(═O)—NR¹³—, —NR¹³—C(═O)—(CH₂)ₘ—, —C(═O)NR¹³—(CH₂)ₘ—(CH₂)ₙ-(Z)_{g}-(CH₂)ₙ, —S—, —SO—, —SO₂—, —SO₂NR⁷—, —SO₂NR¹⁰—, or -Het-. In a preferred embodiment, -Link- is —O—, —(CH₂)ₙ—, —NR¹³—C(═O)—(CH₂)ₘ—, or —C(═O)NR¹³—(CH₂)ₘ.

Each -CAP is, independently, thiazolidinedione, oxazolidinedione, -heteroaryl-C(═O)N R¹³R¹³, heteroaryl-W, —CN, —O—C(═S)NR¹³R¹³, -(Z)_{g}R¹³, —CR¹⁰((Z)_{g}R¹³)((Z)_{g}R¹³)(Z)_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, a cyclic sugar or oligosaccharide, a cyclic amino sugar, oligosaccharide, —CR¹⁰(—(CH₂)ₘ—R⁹)(—(CH₂)ₘ—R⁹), —N(—(CH₂)ₘ—R⁹)(—(CH₂)ₘ—R⁹), —NR¹³(—(CH₂)ₘ—CO₂R¹³),

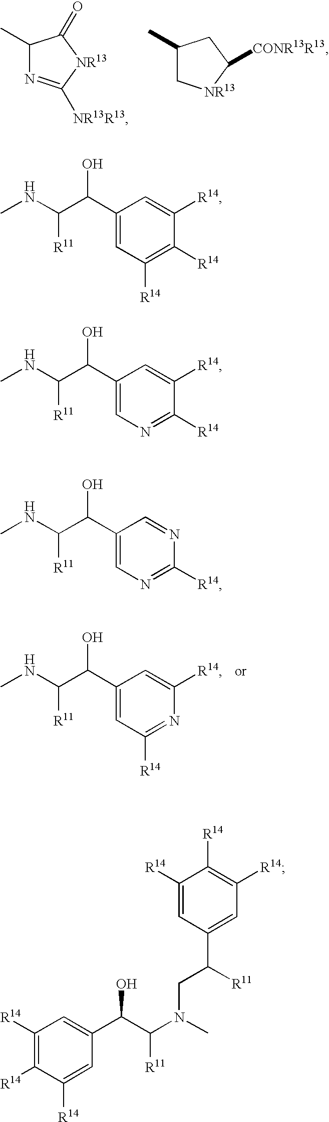

In a preferred embodiment, CAP is

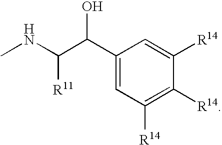

Each Ar is, independently, phenyl, substituted phenyl, wherein the substituents of the substituted phenyl are 1-3 substituents independently selected from the group consisting of OH, OCH₃, NR¹³R¹³, Cl, F, and CH₃, or heteroaryl.

Examples of heteroaryl include pyridinyl, pyrazinyl, furanyl, thienyl, tetrazolyl, thiazolidinedionyl, imidazoyl, pyrrolyl, quinolinyl, indolyl, adeninyl, pyrazolyl, thiazolyl, isoxazolyl, benzimidazolyl, purinyl, isoquinolinyl, pyridazinyl, pyrimidinyl, 1,2,3-triazinyl, 1,2,4-triazinyl, 1,3,5-triazinyl, cinnolinyl, phthalazinyl, quinazolinyl, quinoxalinyl, and pterdinyl groups.

Each W is, independently, thiazolidinedione, oxazolidinedione, heteroaryl-C(═O)N R¹³R¹³, —CN, —O—C(═S)NR¹³R¹³, -(Z)_{g}R¹³, —CR¹⁰((Z)_{g}R¹³)((Z)_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, a cyclic sugar or oligosaccharide, a cyclic amino sugar, oligosaccharide,

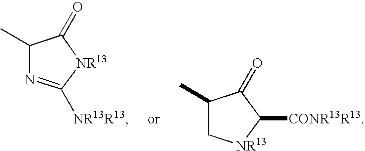

There is at least one R⁵ on A¹ and A² and the remaining substituents are R⁶. Each R⁶ is, independently, R⁵, —R⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—R⁸, —CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose,

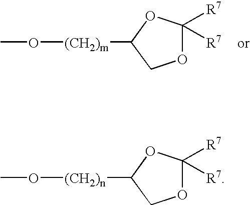

When two R⁶ are —OR¹¹ and are located adjacent to each other on the aromatic carbocycle or aromatic heterocycle, the two OR¹¹ may form a methylenedioxy group; i.e., a group of the formula —O—CH₂—O—.

In addition, one or more of the R⁶ groups can be one of the R⁵ groups which fall within the broad definition of R⁶ set forth above.

R⁶ may be hydrogen. Therefore, provided that the aromatic carbocycle or aromatic heterocycle is substituted with R⁵, the remaining R⁶ may be hydrogen. Preferably, at most, 3 of the R⁶ groups are other than hydrogen. More preferably, provided that the aromatic carbocyle or aromatic heterocycle is substituted with R⁵, then R⁶ is H.

Each g is, independently, an integer from 1 to 6. Therefore, each g may be 1, 2, 3, 4, 5, or 6.

Each m is an integer from 1 to 7. Therefore, each m may be 1, 2, 3, 4, 5, 6, or 7.

Each n is an integer from 0 to 7. Therefore, each n may be 0, 1, 2, 3, 4, 5, 6, or 7.

Each Z is, independently, —(CHOH)—, —C(═O)—, —(CHNR⁷R¹⁰)—, —(C═NR¹⁰)—, —NR¹⁰—, —(CH₂)ₙ—, —(CHNR¹³R¹³)—, —(C═NR¹³)—, or —NR¹³—. As designated by (Z)_{g} in certain embodiments, Z may occur one, two, three, four, five or six times and each occurance of Z is, independently, —(CHOH)—, —C(═O)—, —(CHNR⁷R¹⁰)—, —(C═NR¹⁰)—, —NR¹⁰—, —(CH₂)ₙ—, —(CHNR¹³R¹³)—, —(C═NR¹³)—, or —NR¹³—. Therefore, by way of example and not by way of limitation, (Z)_{g} can be —(CHOH)—(CHNR⁷R¹⁰)—, —(CHOH)—(CHNR⁷R¹⁰)—C(═O)—, —(CHOH)—(CHNR⁷R¹⁰)—C(═O)—(CH₂)ₙ—, —(CHOH)—(CHNR⁷R¹⁰)—C(═O)—(CH₂)ₙ—(CHNR¹³R¹³)—, —(CHOH)—(CHNR⁷R¹⁰)—C(═O)—(CH₂)ₙ—(CHNR¹³R¹³)—C(═O)—, and the like.

In any variable containing —CHOR⁸— or —CH₂OR⁸ groups, when any —CHOR⁸— or —CH₂OR⁸ groups are located 1,2- or 1,3- with respect to each other, the R⁸ groups may, optionally, be taken together to form a cyclic mono- or di-substituted 1,3-dioxane or 1,3-dioxolane.

More specific examples of suitable compounds represented by formula (I) are shown in formulas II and III below wherein A¹ and A² are defined as above:

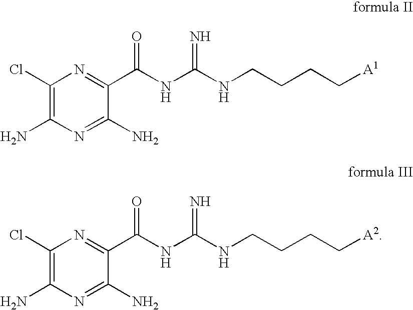

In a preferred aspect of formula II, A¹ is selected from indenyl, napthalenyl, 1,2-dihydronapthalenyl, 1,2,3,4-tetrahydronapthalenyl, anthracenyl, fluorenyl, phenanthrenyl, azulenyl, cyclohepta-1,3,5-trienyl or 5H-dibenzo[a,d]cycloheptenyl.

In another preferred aspect of formula II, A¹ is

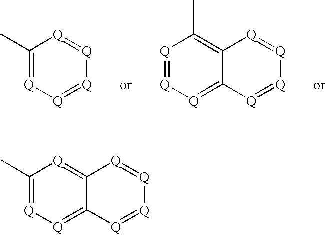

More preferably, R⁵ is —O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

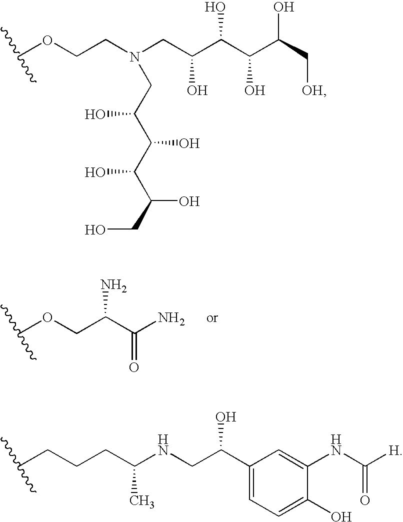

Most preferably, R⁵—O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

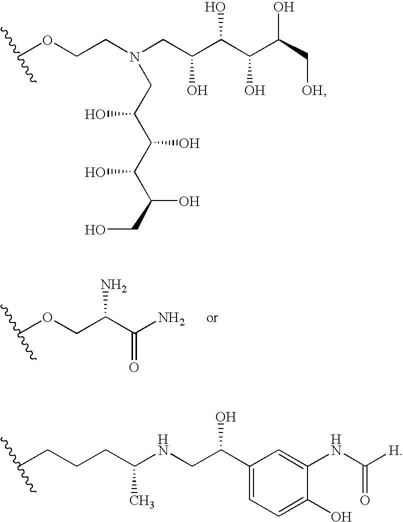

### and six Q are C—H.

In another preferred aspect of formula II, A¹ is

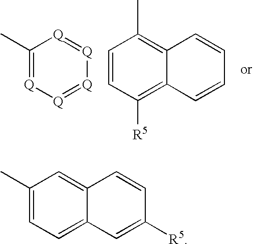

Preferably, R⁵ is —OH, —O—(CH₂)ₘ(Z)_{g}R¹², -Het-(CH₂)ₘ—NH—C(═NR¹³)—NR¹³R¹³, -Het-(CH₂)ₙ-(Z)_{g}-(CH₂)ₘNH—C(═NR¹³)—NR¹³R¹³, -Link-(CH₂)ₘ-(Z)_{g}(CH₂)ₘ-CAP, Link-(CH₂)ₙ—CR¹¹R¹¹-CAP, -Het-(CH₂)ₘ—CONR¹³R¹³, —(CH₂)ₙ—NR^{12 R}¹², —O—(CH₂)ₘNR¹¹R¹¹, —O—(CH₂)ₘ—N⊕—(R¹¹)₃, —(CH₂)ₙ-(Z)_{g}-(CH₂)ₘ—NR¹⁰R¹⁰, -Het-(CH₂)ₘ-(Z)_{g}-NH—C(═NR¹³)—NR¹³R¹³, —O—(CH₂)ₘ(CHOR⁸)(CHOR⁸)ₙ—CH₂OR⁸, —O—(CH₂)ₘ—C(═O)NR⁷R¹⁰, —O—(CH₂)ₘ-(Z)_{g}R⁷, or —O—(CH₂)ₘ—NR¹⁰—CH₂(CHOR⁸)(CHOR⁸)ₙ—CH₂OR⁸. Most preferably, R⁵ is —O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

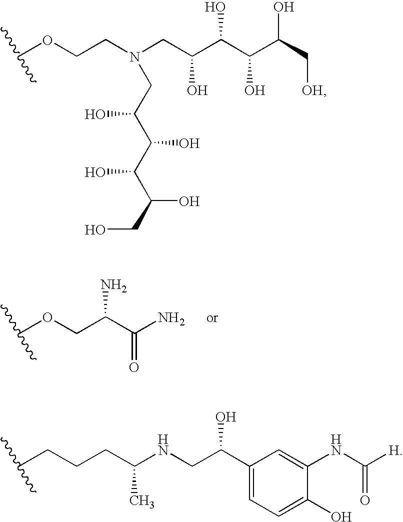

In a preferred aspect of formula III, A² is selected from pyridyl, indolizinyl, indolyl, isoindolyl, indolinyl, benzo[b]furanyl, 2,3-dihydrobenzo[b]furanyl, benzo[b]thiophenyl, 2,3-diydrobenzo[b]thiophenyl, indazolyl, benzimidazolyl, benzthiazolyl, purinyl, quinolinyl, 1,2,3,4-tetrahydroquinolinyl, 3,4-dihydro-2H-chromenyl, 3,4-dihydro-2H-thiochromenyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, phenoxazinyl, dibenzofuranyl, dibenzothiophenyl, 1H-azepinyl, 5H-dibenz[b,f]azepinyl, or 10,11-dihydro-5H-dibenz[b,f]azepinyl.

In another preferred aspect of formula III, A² is

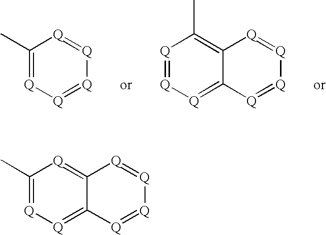

wherein each Q is, independently, C—H, C—R⁵, C—R⁶, or a nitrogen atom, with the proviso that at least one Q is nitrogen and one Q is C—R⁵, and at most three Q in a ring are nitrogen atoms. In a preferred embodiment, only one Q in each ring is nitrogen. In another preferred embodiment, only a single Q is nitrogen. In a particularly preferred embodiment, a single Q is nitrogen, one Q is C—R⁵, and the remaining Q are C—H. In another preferred embodiment, each R⁶ is H. Preferably, R⁵ is —OH, —O—(CH₂)ₘ(Z)_{g}R¹², -Het-(CH₂)ₘ—NH—C(═NR¹³)—NR¹³R¹³, -Het-(CH₂)ₙ-(Z)_{g}-(CH₂)ₘNH—C(═NR¹³)—NR¹³R¹³, -Link-(CH₂)ₘ-(Z)_{g}-(CH₂)ₘ-CAP, Link-(CH₂)ₙ—CR¹¹R¹¹-CAP, -Het-(CH₂)ₘ—CONR¹³R¹³, —(CH₂)ₙ—NR¹²R¹², —O—(CH₂)ₘNR¹¹R¹¹, —O—(CH₂)ₘ—N⊕—(R¹¹)₃, —(CH₂)ₙ-(Z)_{g}-(CH₂)ₘ, —NR¹⁰R¹⁰, -Het-(CH₂)ₘ-(Z)_{g}-NH—C(═NR¹³)—NR¹³R¹³, —O—(CH₂)ₘ(CHOR⁸)(CHOR⁸)ₙ—CH₂OR⁸, —O—(CH₂)ₘ—C(═O)NR⁷R¹⁰, —O—(CH₂)ₘ-(Z)_{g}-R⁷, or —O—(CH₂)ₘ—NR¹⁰—CH₂(CHOR⁸)(CHOR⁸)ₙ—CH₂OR⁸. More preferably, one Q is nitrogen, five Q are C—H and R⁵ is is —OH, —O—(CH₂)ₘ(Z)_{g}R¹², -Het-(CH₂)ₘ—NH—C(═NR¹³)—NR¹³R¹³, -Het-(CH₂)ₙ-(Z)_{g}-(CH₂)ₘNH—C(═NR¹³)—NR¹³R¹³, -Link-(CH₂)ₘ-(Z)_{g}-(CH₂)ₘ-CAP, Link-(CH₂)ₙ—CR¹¹R¹¹-CAP, -Het-(CH₂)ₘ—CONR¹³R¹³, —(CH₂)ₙ—NR¹²R¹², —O(CH₂)ₘNR¹¹R¹¹, —O—(CH₂)ₘ—N⊕—(R¹¹)₃, —(CH₂)ₙ-(Z)_{g}-(CH₂)ₘ—NR¹⁰OR¹⁰, -Het-(CH₂)ₘ-(Z)_{g}NH—C(═NR¹³)—NR¹³R¹³, —O—(CH₂)ₘ(CHOR⁸)(CHOR⁸)ₙ—CH₂OR⁸, —O—(CH₂)ₘ—C(═O)NR⁷R¹⁰, —O—(CH₂)ₘ-(Z)_{g}-R⁷, or —O—(CH₂)ₘ—NR¹⁰—CH₂(CHOR⁸)(CHOR⁸)ₙ—CH₂OR⁸. More preferably, R⁵ is —O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

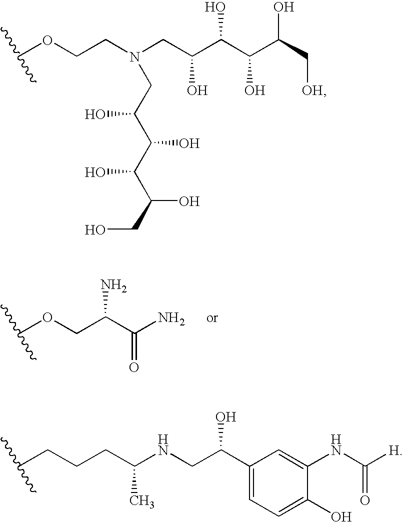

Most preferably, R⁵ is —O—CH₂—(CHOH)—CH₂OH, —OH, —O—(CH₂)₃NH₂, —O—(CH₂)₃NH(C═NH)NH₂, —O—(CH₂)₂NH(C═NH)NH₂, —O—CH₂(CO)NH₂, —O—(CH₂)₂—N⊕—(CH₃)₃,

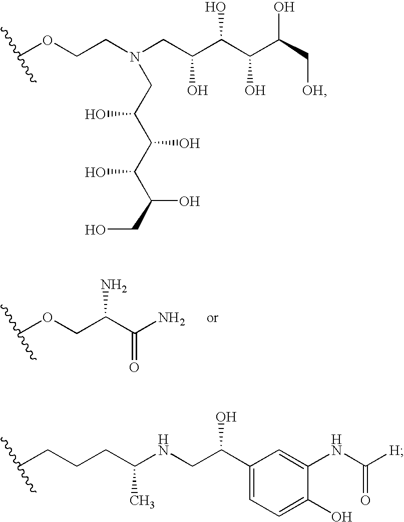

a single Q is nitrogen and five Q are C—H.

In a particularly preferred embodiment, the compounds of formula I, formula II, or formula III are:

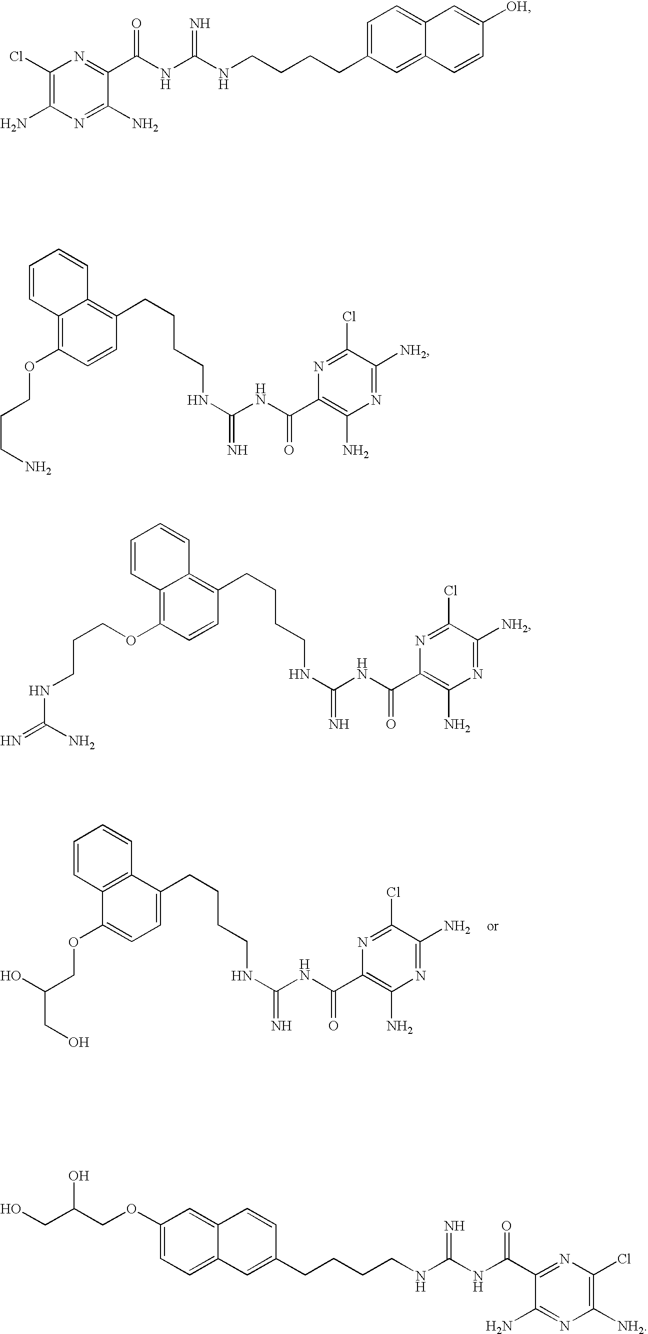

The compounds described herein may be prepared and used as the free base. Alternatively, the compounds may be prepared and used as a pharmaceutically acceptable salt. Pharmaceutically acceptable salts are salts that retain or enhance the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid and the like; and (c) salts formed from elemental anions for example, chlorine, bromine, and iodine.

It is to be noted that all enantiomers, diastereomers, and racemic mixtures, tautomers, polymorphs, pseudopolymorphs and pharmaceutically acceptable salts of compounds within the scope of formula (I), formula II, or formula III are embraced by the present invention. All mixtures of such enantiomers and diastereomers are within the scope of the present invention.

A compound of formula I-III and its pharmaceutically acceptable salts may exist as different polymorphs or pseudopolymorphs. As used herein, crystalline polymorphism means the ability of a crystalline compound to exist in different crystal structures. The crystalline polymorphism may result from differences in crystal packing (packing polymorphism) or differences in packing between different conformers of the same molecule (conformational polymorphism). As used herein, crystalline pseudopolymorphism means the ability of a hydrate or solvate of a compound to exist in different crystal structures. The pseudopolymorphs of the instant invention may exist due to differences in crystal packing (packing pseudopolymorphism) or due to differences in packing between different conformers of the same molecule (conformational pseudopolymorphism). The instant invention comprises all polymorphs and pseudopolymorphs of the compounds of formula I-III and their pharmaceutically acceptable salts.

A compound of formula I-III and its pharmaceutically acceptable salts may also exist as an amorphous solid. As used herein, an amorphous solid is a solid in which there is no long-range order of the positions of the atoms in the solid. This definition applies as well when the crystal size is two nanometers or less. Additives, including solvents, may be used to create the amorphous forms of the instant invention. The instant invention comprises all amorphous forms of the compounds of formula I-III and their pharmaceutically acceptable salts.

The compounds of formula I-III may exist in different tautomeric forms. One skilled in the art will recognize that amidines, amides, guanidines, ureas, thioureas, heterocycles and the like can exist in tautomeric forms. By way of example and not by way of limitation, compounds of formula I-III can exist in various tautomeric forms as shown below:

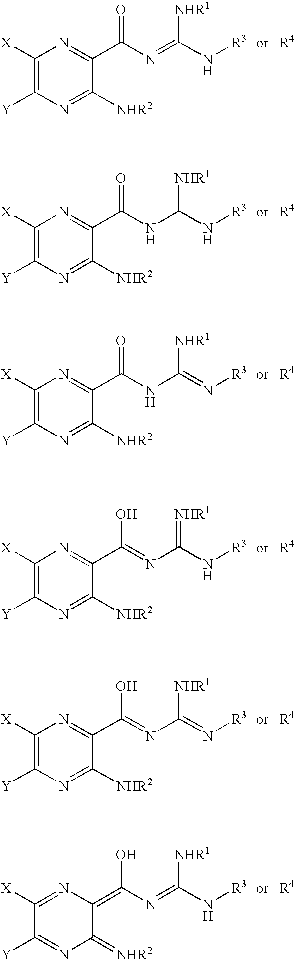

All possible tautomeric forms of the amidines, amides, guanidines, ureas, thioureas, heterocycles and the like of all of the embodiments of formula I-III are within the scope of the instant invention.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l, D and L, or (+) and (−) are employed to designate the sign of rotation of plane-polarized light by the compound, with S, (−), or l meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stercoselection or stereospecificity in a chemical reaction or process. The terms “racemic mixture” and “racemate” refer to an equimolar mixture of two enantiomeric species, devoid of optical activity.

A single stereoisomer, e.g. an enantiomer, substantially free of its stereoisomer may be obtained by resolution of the racemic mixture using a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

In another preferred embodiment of the present invention the compound of Formula I is represented by the formula:

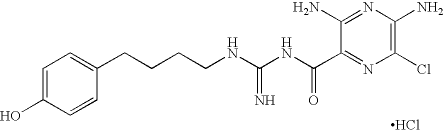

In another preferred embodiment of the present invention the compound of Formula I is represented by the formula:

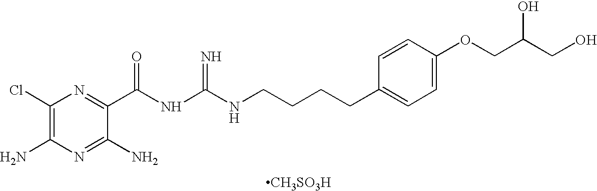

In another preferred embodiment of the present invention the compound of Formula I is represented by the formula:

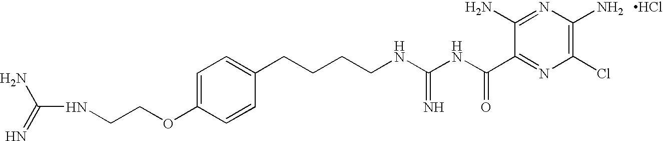

In another preferred embodiment of the present invention the compound of Formula I is represented by the formula:

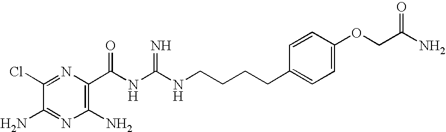

In another preferred embodiment of the present invention the compound of Formula I is represented by the formula:

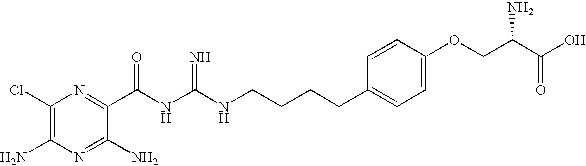

In another preferred embodiment of the present invention the compound of Formula I is represented by the formula:

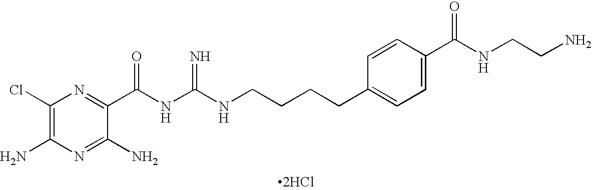

In another preferred embodiment of the present invention the compound of Formula I is represented by the formula:

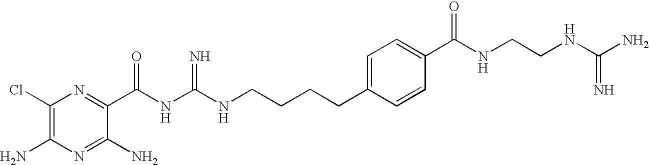

In another preferred embodiment of the present invention the compound of Formula I is represented by the formula:

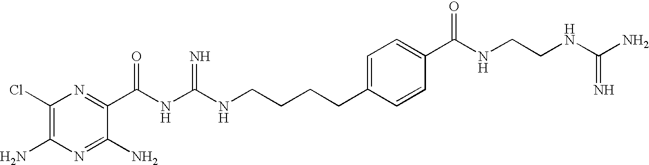

In another preferred embodiment of the present invention the compound of Formula I is represented by the formula:

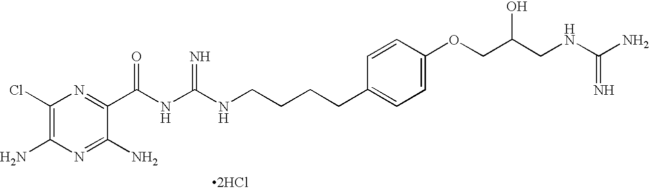

In another preferred embodiment of the present invention the compound of Formula I is represented by the formula:

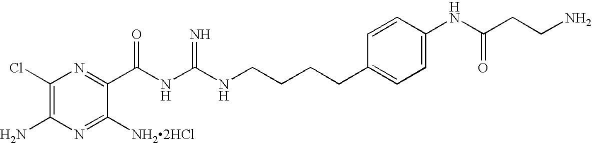

In another preferred embodiment of the present invention the compound of Formula I is represented by the formula:

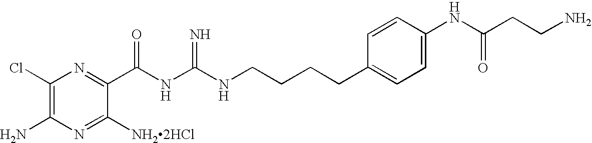

The compounds of formula (I) may be prepared and used as the free base or zwiterion. Alternatively, the compounds may be prepared and used as a pharmaceutically acceptable salt. Pharmaceutically acceptable salts are salts that retain or enhance the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid and the like; and (c) salts formed from elemental anions for example, chlorine, bromine, and iodine.

It is to be noted that all enantiomers, diastereomers, and racemic mixtures of compounds within the scope of formula (I) are embraced by the present invention. All mixtures of such enantiomers and diastereomers are within the scope of the present invention. The above compounds can be a pharmaceutically acceptable salt thereof, and wherein the above compounds are inclusive of all racemates, enantiomers, diastereomers, tautomers, polymorphs and pseudopolymorphs thereof.

Without being limited to any particular theory, it is believed that the compounds of formula (I) function in vivo as ASICs blockers. By blocking ASIC channels present in the body the compounds of formula (I) the tissue would experience reduced acid mediated pain and inflammation.

The present invention also provides methods of treatment that take advantage of the properties of the compounds of formula (I) discussed above. Thus, subjects that may be treated by the methods of the present invention include, but are not limited to, patients afflicted with angina, stroke, ischemic heart disease, arthritis, cancer, infections, inflammation, and traumatic injuries, gastrointestinal disorders, oropharangeal disease and damage, acute and chronic cough, central nervous system disorders and psychiatric diseases or manifestations such as memory loss, learning disabilities, fear and anxiety.

The present invention is concerned primarily with the treatment of human subjects, but may also be employed for the treatment of other mammalian subjects, such as dogs and cats, for veterinary purposes.

As discussed above, the compounds used to prepare the compositions of the present invention may be in the form of a pharmaceutically acceptable free base. Because the free base of the compound is generally less soluble in aqueous solutions than the salt, free base compositions are employed to provide more sustained release of active agent. An active agent in particulate form which has not dissolved into solution is not available to induce a physiological response, but serves as a depot of bioavailable drug which gradually dissolves into solution.

Another aspect of the present invention is a pharmaceutical composition, comprising a compound of formula (I) in a pharmaceutically acceptable carrier (e.g., an aqueous carrier solution

Another aspect of the present invention is a pharmaceutical formulation, comprising an active compound as described above in a pharmaceutically acceptable carrier (e.g., an aqueous carrier solution). In general, the active compound is included in the composition in an amount effective to block ASIC channels.

The active compounds disclosed herein may be administered to mucosal surfaces by any suitable means, including topically, orally, rectally, vaginally, ocularly, dermally, intravenously, by inhalation, etc. For example, for the treatment of GERD induced pain, the active compounds may be administered orally. The active compound may be combined with a pharmaceutically acceptable carrier in any suitable form, such as sterile physiological or dilute saline or topical solution or a carrier to maintain it at a site ‘sticky vehicle’. Excipients may be included in the formulation to enhance the solubility of the active compounds, as desired.

Solid or liquid particulate active agents prepared for practicing the present invention could, as noted above, include particles of respirable or non-respirable size; that is, for respirable particles, particles of a size sufficiently small to pass through the mouth and larynx upon inhalation and into the bronchi and alveoli of the lungs, and for non-respirable particles, particles sufficiently large to be retained in the nasal airway passages rather than pass through the larynx and into the bronchi and alveoli of the lungs. In general, particles ranging from about 1 to 5 microns in size (more particularly, less than about 4.7 microns in size) are respirable. Particles of non-respirable size are greater than about 5 microns in size, up to the size of visible droplets. Thus, for nasal administration, a particle size in the range of 10-500 μm may be used to ensure retention in the nasal cavity.

In the manufacture of a formulation according to the invention, active agents or the physiologically acceptable salts or free bases thereof are typically admixed with, inter alia, an acceptable carrier. Of course, the carrier must be compatible with any other ingredients in the formulation and must not be deleterious to the patient. The carrier must be solid or liquid, or both, and is preferably formulated with the compound as a unit-dose formulation, for example, a capsule, that may contain 0.5% to 99% by weight of the active compound. One or more active compounds may be incorporated in the formulations of the invention, which formulations may be prepared by any of the well-known techniques of pharmacy consisting essentially of admixing the components.

Compositions containing respirable or non-respirable dry particles of micronized active agent may be prepared by grinding the dry active agent with a mortar and pestle, and then passing the micronized composition through a 400 mesh screen to break up or separate out large agglomerates.

The particulate active agent composition may optionally contain a dispersant which serves to facilitate the formulation of an aerosol. A suitable dispersant is lactose, which may be blended with the active agent in any suitable ratio (e.g., a 1 to 1 ratio by weight).

Active compounds disclosed herein may be administered to airway surfaces including the nasal passages, sinuses and lungs of a subject by a suitable means know in the art, such as by nose drops, mists, etc. In one embodiment of the invention, the active compounds of the present invention and administered by transbronchoscopic lavage. In a preferred embodiment of the invention, the active compounds of the present invention are deposited on lung airway surfaces by administering an aerosol suspension of respirable particles comprised of the active compound, which the subject inhales. The respirable particles may be liquid or solid. Numerous inhalers for administering aerosol particles to the lungs of a subject are known.

The dosage of the active compounds disclosed herein will vary depending on the condition being treated and the state of the subject, but generally may be from about 0.01, 0.03, 0.05, 0.1 to 1, 5, 10 or 20 mg of the pharmaceutic agent, delivered orally, topically, rectally, intravenously or by inhalation. The daily dose may be divided among one or multiple unit dose administrations.

In one embodiment of the invention, the particulate active agent composition may contain both a free base of active agent and a pharmaceutically acceptable salt to provide both early release and sustained release of active agent for dissolution into the mucus secretions of the nose. Such a composition serves to provide both early relief to the patient, and sustained relief over time. Sustained relief, by decreasing the number of daily administrations required, is expected to increase patient compliance with the course of active agent treatments.

The compounds of formula (I) may be synthesized according to procedures known in the art. A representative synthetic procedure is shown in the scheme below:

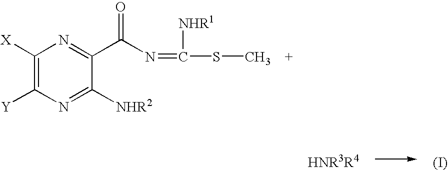

These procedures are described in, for example, E. J. Cragoe, “The Synthesis of Amiloride and Its Analogs” (Chapter 3) in Amiloride and Its Analogs, pp. 25-36, incorporated herein by reference. Other methods of preparing the compounds are described in, for example, U.S. Pat. No. 3,313,813, incorporated herein by reference. See in particular Methods A, B, C, and D described in U.S. Pat. No. 3,313,813, incorporated herein by reference. Other methods useful for the preparation of these compounds, especially for the preparation of the novel HNR³R⁴ fragments are described in, for example, the patents and applications cited above.

Several assays may be used to characterize the compounds of the present invention. A representative assay is discussed below (Kellenberger and Schild Physiol Rev. 2002), incorporated herein by reference.

In Vitro Measure of ASIC Blocking Activity

One assay used to assess the mechanism of action and/or potency of the compounds of the present invention involves the determination of drug inhibition of ASIC H⁺-sensitive current in oocytes overexpressing ASICs channels (ASICs 1A) using the patch clamp method. ASIC1a Expression—Complementary cDNA of the human ASIC1a was subcloned in the pSDEasy cloning vector for in vitro transcription and expression in Xenopus oocytes. Only stage V and VI Xenopus oocytes were injected with 5 ng of cRNA encoding hASIC1a and used in the experiments.

Electrophysiology—Electrophysiological measurements were performed 24-36 h after oocyte injection with ASIC cRNA. Macroscopic ASIC currents (IASIC) were elicited every 30 s by rapid changes in extracellular pH from 7.4 to 6.0 and were measured using the two electrode voltage clamp for whole-cell currents. The bathing solution contained (in mM) NaCl 120, MgCl₂ 2,HEPES 10, adjusted to pH 7.5 with NaOH. Changes in extracellular pH were achieved using the same bathing solution buffered at pH 6.0. The cut-open configuration of the Xenopus oocyte allows the recording of macroscopic ASIC currents while continuously perfusing inside and outside of the oocytes. A microperfusion pipette, in which two thin capillaries (Microfil, World Precision instruments) had been inserted, was used for the intracellular perfusion and served as an intracellular electrode potential measurement. The intracellular solution contained (in mM) potassium gluconate 90, KCl 10, sodium gluconate 2, MgCl2 1, BAPTA 0.2, HEPES-N-methyl-D-glucamine 10, adjusted to pH 7.35. Methanethiosulfonates or Cd2 (1 mM) were added to the solution. The holding potential was 100 mV. The extracellular solution corresponded to the bathing solution in the two-electrode voltage clamp experiments.

### EXAMPLES

Having generally described this invention, a further understanding can be obtained by reference to certain specific examples which are provided herein for purposes of illustration only and are not intended to be limiting unless otherwise specified.

Dose-effect relationships for all compounds are presented in Example 1 based on normalized ASIC1a current. IC₅₀ values are calculated and compared to amiloride as positive controls Example 2.

### Example 1

### The Effects of Amiloride, 608, 522, 518, 765, 643 on ASIC Current Generated by Oocytes Overexpressing the 1A Subunit

### Example 2

### The IC₅₀ of Amiloride, 518, 522, 608, 643 and 765 on ASIC1 A Current

| | IC50 | |
|---|---|---|
| Compound | (μM) | SEM |
| Amiloride | 60.3 | 7 |
| 518:01 | 4.41 | 0.96 |
| 522:01 | 1.27 | 0.32 |
| 608:03 | 1.75 | 0.26 |
| 643:03 | 3.3 | 0.57 |
| 765:15 | 3.75 | 0.43 |

One other method for measuring potency of ASIC blockers is by using an automated patch-clamping apparatus, the QPatch 16. The QPatch 16 uses four pipette heads that afford more efficient assays and faster throughput for ion channel drug discovery. The QPlate contains 16 individual patch-clamp sites that are operated asynchronously and in parallel. Ringer's solutions and compounds are applied by four pipettes. HEK-293 cells expressing ASIC ion channels are kept in culture medium in the stirred reservoir for up to four hours. Prior to testing, the cells were transferred to an on-board mini centrifuge, spun down and washed in Ringer's solution twice before being applied to the pipetting wells in the QPlate. Gigaseals were formed upon execution of a combined suction/voltage protocol. Further suction lead to whole-cell configuration. Solutions and compounds were applied through the glass flow channels in the QPlate. All currents were recorded at a patch potential of −70 mV. Liquid flow was laminar with exchange time constants in of 50-100 ms.

1. A method of treating one of more conditions selected from the group consisting of pain, ischemic pain due to cardiovascular disease, stroke-induced neural damage, pain due to arthritis, ischemic pain due to cancer, pain due to inflammation, pain due to infection, pain due to infection, ischemic pain due to oropharengeal diseases or damage, ischemic pain due to traumatic injuries, acute and chronic cough, pain due to gastrointestinal disorders, central nervous system disorders, psychiatric diseases or manifestations, comprising administering an effective amount of a compound of formula I to a subject in need thereof: 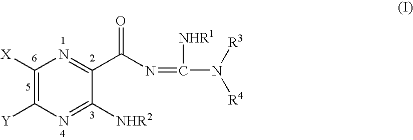
and racemates, enantiomers, diastereomers, tautomers, polymorphs, pseudopolymorphs and pharmaceutically acceptable salts, thereof, wherein:
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘR⁸, —(CH₂CH₂O)ₘ—CH₂CH₂NR⁷R₁₀, —(CH₂)ₙ—C(═O)NR⁷R¹⁰, 13 (CH₂)ₙ-(Z)_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 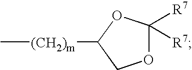
R³ and R⁴ are each, independently, hydrogen, lower alkyl, hydroxyl-lower alkyl, phenyl, (phenyl)-lower alkyl, (halophenyl)-lower alkyl, ((lower-alkyl)phenyl)-lower-alkyl, ((lower-alkoxy)phenyl)-lower-alkyl, (naphthyl)-lower-alkyl, or (pyridyl)-lower-alkyl, or a group represented by formula A or formula B, with the proviso that at least one of R³ and R⁴ is a group represented by the formula A or formula B;
—(C(R^{L})₂)ₒ-x-(C(R^{L})₂)ₚA¹ formula A:
—(C(R^{L})₂)ₒ-x-(C(R^{L})₂)ₚA² formula B:
A¹ is a C₆-C₁₅-membered aromatic carbocycle substituted with at least one R⁵ and the remaining substituents are R⁶;
A² is a six to fifteen-membered aromatic heterocycle substituted with at least one R⁵ and the remaining substituents are R⁶ wherein said aromatic heterocycle comprises 1-4 heteroatoms selected from the group consisting of O, N, and S;
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, -O-glucose, 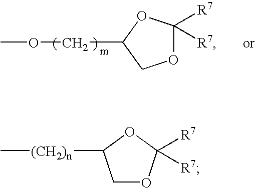
each o is, independently, an integer from 0 to 10;
each p is, independently, an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or a single bond;
each R⁵ is, independently, OH, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}R⁷, —O—(CH₂)ₘ-(Z)_{g}R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 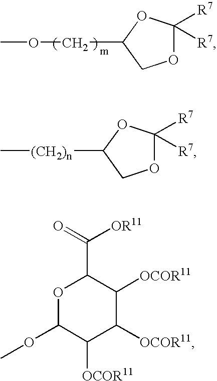
—(CH₂)ₙ—CO₂R¹³, -Het-(CH₂)ₘ—CO₂R¹³, —(CH₂)ₙ-(Z)_{g}—CO₂R¹³, -Het-(CH₂)ₘ-(Z)_{g}—CO₂R¹³, (CH₂)ₙ—NR¹⁰—(CH₂)ₘ(CHOR⁸)ₙ—CO₂R¹³, -Het-(CH₂)ₘ—NR¹⁰—(CH₂)ₘ(CHOR⁸)ₙ—CO₂R¹³, —(CH₂)ₙ—(CHOR⁸)ₘ—CO₂R¹³, -Het-(CH₂)ₘ—(CHOR⁸)ₘ—(CO₂R¹³, —(CH₂)ₙ—(CHOR⁸)ₘ(Z)_{g}-CO₂R¹³, -Het-(CH₂)ₙ—(CHOR⁸)ₘ-(Z)_{g}-CO₂R¹³, —(CH₂)ₙ-(Z)_{g}-(CH₂)ₘ—CO₂R¹³, —(CH₂)ₙ-(Z)_{g}-(CH₂)ₘ—CO₂R¹³, —(CH₂)ₙ-(Z)_{g}(CHOR⁸)ₘ-(Z)_{g}—CO₂R¹³, -Het-(CH₂)ₙ-(Z)_{g}-(CHOR⁸)ₘ-(Z)_{g}-CO₂R¹³, —(CH₂)ₙ—CONH—C(═NR¹³)—NR¹³R¹³, -Het-(CH₂)ₙ—CO—NH—C(═NR¹³)—NR¹³R¹³, —(CH₂)ₙ-(Z)_{g}-CONH—C(═NR¹³)—NR¹³R¹³, -Het-(CH₂)ₙ-(Z)_{g}-CONH—C(═NR¹³)—NR¹³R¹³, —(CH₂)ₙ—NR¹⁰—(CH₂)ₘ(CHOR⁸)ₙ—CONH—C(═NR¹³)—NR¹³R¹³, -Het-(CH₂)ₙ—NR¹⁰—(CH₂)ₘ(CHOR⁸)ₙ—CONH—C(═NR¹³)—NR¹³R¹³, —(CH₂)ₙ—(CHOR⁸)ₘ—CONH—C(═NR¹³)—NR¹³R¹³, -Het-(CH₂)ₙ—(CHOR⁸)ₘ—CONH—C(═NR¹³)—NR¹³R¹³, (CH₂)ₙl(CHOR⁸)ₘ-(Z)_{g}-CONH—C(═NR¹³)—NR¹³R¹³, -Het-(CH₂)ₙ—(CHOR⁸)ₘ-(Z)_{g}-CONH—C(═NR¹³)—NR¹³R¹³, —(CH₂)ₙ-(Z)_{g}-(CH₂)ₘCONH—C(═NR¹³)—NR¹³R¹³, -Het-(CH₂)ₙ-(Z)_{g}-(CH₂)ₘCONH—C(═NR¹³)—NR¹³R¹³, —(CH₂)ₙ-(Z)_{g}-(CHOR⁸)ₘ-(Z)_{g}-CONH—C(═NR¹³)—NR¹³R¹³, Het-(CH₂)ₙ-(Z)_{g}-(CHOR⁸)ₘ-(Z)_{g}-CONH—C(═NR¹³)—NR¹³R¹³, —(CH₂)ₙ—CONR⁷—CONR¹³R¹³, -Het-(CH₂)ₙ—CONR⁷—CONR¹³R¹³, —(CH₂)ₙ-(Z)_{g}—CONR⁷—CONR¹³R¹³, —(CH₂)ₙ-(Z)_{g}-CONR⁷—CONR¹³R¹³, —(CH₂)ₙ—NR¹⁰—(CH₂)ₘ(CHOR⁸)ₙ—CONR⁷—CONR¹³R¹³, -Het-(CH₂)ₙ—NR¹⁰—(CH₂)ₘ(CHOR⁸)ₙ—CONR⁷—CONR¹³R¹³, —(CH₂)ₙ—(CHOR⁸)ₘ—CONR⁷—CONR¹³R¹³, Het-(CH₂)ₙ—(CHOR⁸)ₘ—CONR⁷—CONR¹³R¹³, —(CH₂)ₙ—(CHOR⁸)ₘ-(Z)_{g}-CONR⁷—CONR¹³R¹³, -Het-(CH₂)ₙ—(CHOR⁸)ₘ-(Z)_{g}-CNR⁷—CONR¹³R¹³, —(CH₂)ₙ-(Z)_{g}-(CH₂)ₘCONR⁷—CONR¹³R¹³, -Het-(CH₂)ₙ-(Z)_{g}-(CH₂)ₘCONR⁷—CONR¹³R¹³, —(CH₂)ₙ(Z)_{g}(CHOR⁸)ₘ-(Z)_{g}-CONR⁷—CONR¹³R¹³, -Het-(CH₂)ₙ-(Z)_{g}(CHOR⁸)ₘ-(Z)_{g}-CONR⁷-CONR¹³R¹³, —(CH₂)ₙ—CONR⁷SO₂NR¹³R¹³, -Het-(CH₂)ₘ—CONR⁷SO₂NR¹³R¹³, —(CH₂)ₙ-(Z)_{g}-CONR⁷SO₂NR¹³R¹³, -Het-(CH₂)ₘ-(Z)_{g}-CONR⁷SO₂NR¹³R¹³, —(CH₂)ₙ—NR¹⁰—(CH₂)ₘ(CHOR⁸)ₙ—CONR⁷SO₂NR¹³R¹³, -Het-(CH₂)ₘ—NR¹⁰—(CH₂)ₘ(CHOR⁸)ₘ—CONR⁷SO₂NR¹³R¹³, —(CH₂)ₙ—(CHOR⁸)ₘCONR⁷SO₂NR¹³R¹³, -Het-(CH₂)_{m—(CHOR}⁸)ₘ—CONR⁷SO₂NR¹³R¹³, —(CH₂)ₙ—(CHOR⁸)ₘ-(Z)_{g}-CONR⁷SO₂NR¹³R¹³, -Het-(CH₂)ₙ—(CHOR⁸)ₘ-(Z)_{g}-CONR⁷SO₂NR¹³R¹³, —(CH₂)ₙ-(Z)_{g}-(CH₂)ₘCONR⁷SO₂NR¹³R¹³, -Het-(CH₂)ₙ-(Z)_{g}-(CH₂)ₘCONR⁷SO₂NR¹³R¹³, —(CH₂)ₙ-(Z)_{g}-(CHOR⁸)ₘ-(Z)_{g}-CONR⁷SO₂NR¹³R¹³, -Het-(CH₂)ₙ-(Z)_{g}-(CHOR⁸)ₘ-(Z)_{g}-CONR⁷SO₂NR¹³R¹³, —(CH₂)ₙ-SO₂NR¹³R¹³, -Het-(CH₂)ₘ—SO₂NR¹³R¹³, —(CH₂)ₙ-(Z)_{g}-SO₂NR¹³R¹³, -Het-(CH₂)ₙ, -(Z)_{g}-SO₂NR¹³R¹³, —(CH₂)ₙ—NR¹⁰—(CH₂)ₘ(CHOR⁸)ₙ—SO₂NR¹³R¹³, -Het-(CH₂)ₘ—NR¹⁰—(CH₂)ₘ(CHOR⁸)ₙ—SO₂NR¹³R¹³ , —(CH₂)ₙ—(CHOR⁸)ₘ—-SO₂NR¹³R¹³, -Het-(CH₂)ₘ—(CHOR⁸)ₘ—SO₂NR¹³R¹³, —(CH₂)ₘ—(CHOR⁸)ₘ-(Z)_{g}-SO₂NR¹³R¹³, -Het-(CH₂)ₙ—(CHOR⁸)ₘ-(Z)_{g}-SO₂NR¹³R¹³, —(CH₂)ₙ-(Z)_{g}-(CH₂)ₘSO₂NR¹³R¹³, -Het-(CH₂)ₙ-(Z)_{g}-(CH₂)ₘSO₂NR¹³R¹³, —(CH₂)ₙ-(Z)_{g}-(CHOR⁸)ₙ-(Z)_{g}-SO₂NR¹³R¹³, -Het-(CH₂)ₙ-(Z)_{g}-(CHOR⁸)ₘ-(Z)_{g}-SO₂NR¹³R¹³, —(CH₂)ₙ—CONR¹³R¹³, -Het-(CH₂)ₘ—CONR¹³R¹³, —(CH₂)ₙ-(Z)_{g}-CONR¹³R¹³, -Het-(CH₂)ₘ-(Z)_{g}-CONR¹³R¹³, —(CH₂)ₙ—NR¹⁰)—(CH₂)ₘ(CHOR⁸)ₙ—CONR¹³R¹³, -Het-(CH₂)ₘ—NR¹⁰—(CH₂)ₘ(CHOR⁸)ₙ—CONR¹³R¹³, —(CH₂)ₙ—(CHOR⁸)ₘ—CONR¹³R¹³, -Het-(CH₂)ₘ—(CHOR⁸)ₘ—CONR¹³R¹³, —(CH₂)ₙ—(CHOR⁸)ₘ-(Z)_{g}-CONR¹³R¹³, -Het-(CH₂)ₙ—(CHOR⁸)ₘ-(Z)_{g}-CONR¹³R¹³, —(CH₂)ₙ-(Z)_{g}-(CH₂)ₘCONR¹³R¹³, -Het-(CH₂)ₙ-(Z)_{g}-(CH₂)ₘ—CONR¹³R¹³, —(CH₂)ₙ-(Z)_{g}-(CHOR⁸)ₘ-(Z)_{g}—CONR¹³R¹³, -Het-(CH₂)ₙ-(Z)_{g}-(CHOR⁸)ₘ-(Z)_{g}-CONR¹³R¹³, —(CH₂)ₙ—CONR⁷COR¹³, -Het-(CH₂)ₘ—CONR⁷COR¹³, —(CH₂)ₙ-(Z)_{g}-CONR⁷COR¹³, -Het-(CH₂)ₘ-(Z)_{g}-CONR⁷COR¹³, —(CH₂)ₙ—NR¹⁰—(CH₂)ₘ(CHOR⁸)ₙ—CONR⁷COR¹³, -Het-(CH₂)ₘ—NR¹⁰—(CH₂)ₘ(CHOR⁸)ₙ—CONR⁷COR¹³, —(CH₂)ₙ—(CHOR⁸)ₘ—CONR⁷COR¹³, -Het-(CH₂)ₘ—(CHOR⁸)ₘ—CONR⁷COR¹³, —(CH₂)ₙ—(CHOR⁸)ₘ-(Z)_{g}-CONR⁷COR¹³, -Het-(CH₂)ₙ—(CHOR⁸)ₘ-(Z)_{g}-CONR⁷COR¹³, —(CH₂)ₙ-(Z)_{g}-(CH₂)ₘCONR⁷COR¹³, —(CH₂)ₙ-(Z)_{g}(CH₂)ₘCONR⁷COR¹³, -Het-(CH₂)ₙ-(Z)_{g}(CHOR⁸)ₘ-(Z)_{g}-CONR⁷COR¹³, —(CH₂)ₙ—CONR⁷CO₂R¹³, —(CH₂)ₙ-(Z)_{g}-CONR⁷CO₂R¹³, -Het-(CH₂)ₘ-(Z)_{g}-CONR⁷CO₂R¹³, —(CH₂)ₙ—NR¹⁰—(CH₂)ₘ(CHOR⁸)ₙ—CONR⁷CO₂R¹³, -Het-(CH₂)ₘ—NR¹⁰—(CH₂)ₘ(CHOR⁸)ₙ—CO NR⁷CO₂R¹³, —(CH₂)ₙ—(CHOR⁸)ₘ—CONR⁷CO₂R¹³, -Het-(CH₂)ₘ—(CHOR⁸)ₘ—CONR⁷CO₂R¹³, —(CH₂)ₙ—(CHOR⁸)ₘ-(Z)_{g}- CONR⁷CO₂R¹³, -Het-(CH₂)ₙ—(CHOR⁸)ₘ-(Z)_{g}-CONR⁷CO₂R¹³, —(CH₂)ₙ-(Z)_{g}-(CH₂)ₘCONR⁷CO₂R¹³, -Het-(CH₂)ₙ-(Z)_{g}-(CH₂)ₘCONR⁷CO₂R¹³, —(CH₂)ₙ-(Z)_{g}-(CHOR⁸)ₘ-(Z)_{g}—CONR⁷CO₂R¹³, -Het-(CH₂)ₙ-(Z)_{g}-(CHOR⁸)ₘ-(Z)_{g}-CONR⁷CO₂R¹³, —(CH₂)ₙ—NH—C(═NR¹³)—NR¹³R¹³, -Het-(CH₂)ₘ—NH—C(═NR¹³)—NR¹³R¹³, —(CH₂)ₙ-(Z)_{g}-NH—C(═NR¹³)—NR¹³R¹³, -Het-(CH₂)ₘ-(Z)_{g}-NH—C(═NR¹³)—NR¹³R¹³, —(CH₂)ₙ—NR¹⁰—(CH₂)ₘ(CHOR⁸)ₙ—NH—C(═NR¹³)—NR¹³R¹³, -Het-(CH₂)ₘ—NR¹⁰—(CH₂)ₘ(CHOR⁸)ₙ—NH—C(═NR¹³)—NR¹³R¹³, —(CH₂)ₙ—(CHOR⁸)ₘ—NH—C(═NR¹³)—NR¹³R¹³, -Het-(CH₂)ₘ—(CHOR⁸)ₘ—NH—C(═NR¹³)—NR¹³R¹³, (CH₂)ₙ—(CHOR⁸)ₘ-(Z)_{g}-NH—C(═NR¹³)—NR¹³R¹³, -Het-(CH₂)ₙ—(CHOR⁸)ₘ-(Z)_{g}-NH—C(═NR¹³)—NR¹³R¹³, —(CH₂)ₙ-(Z)_{g}-(CH₂)ₘNH—C(═NR¹³)—NR¹³R¹³, -Het-(CH₂)ₙ-(Z)_{g}-(CH₂)ₘNH—C(═NR¹³)—NR¹³R¹³, —(CH₂)ₙ-(Z)_{g}-(CHOR⁸)ₘ-(Z)_{g}-NH—C(═NR¹³)—NR¹³R¹³, -Het-(CH₂)ₙ-(Z)_{g}-(CHOR⁸)ₘ-(Z)_{g}-NH—C(═NR¹³)—NR¹³R¹³, —(CH₂)ₙ—C(═NR¹³)—NR¹³R¹³, Het-(CH₂)ₘ—C(═NH)—NR¹³R¹³, —(CH₂)ₙ-(Z)_{g}-C(═NH)—NR¹³R¹³, Het-(CH₂)ₘ-(Z)_{g}-C(═NH)—NR¹³R¹³, —(CH₂)ₙ—NR¹⁰—(CH₂)ₘ(CHOR⁸)ₙ—C(═NR¹³)—NR¹³R¹³, Het-(CH₂)ₘ—NR¹⁰—(CH₂)ₘ(CHOR⁸)ₙ—C(═NR¹³)—NR¹³R¹³, —(CH₂)ₙ—(CHOR⁸)ₘ—C(═NR¹³)—NR¹³R¹³, -Het-(CH₂)ₘ—(CHOR⁸)ₘ—C(═NR¹³)—NR¹³R¹³, —(CH₂)ₙ—(CHOR⁸)ₘ-(Z)_{g}-C(═NR¹³)—NR¹³R¹³, -Het-(CH₂)ₙ—(CHOR⁸)ₘ-(Z)_{g}-C(═NR¹³)—NR¹³R¹³, —(CH₂)ₙ-(Z)_{g}-(CH₂)ₘ—C(═NHC(═NR¹³)—NR¹³R¹³, Het-(CH₂)ₙ-(Z)_{g}-(CH₂)ₘ—C(═N R¹³)—NR¹³R¹³, —(CH₂)ₙ-(Z)_{g}-(CHOR⁸)ₘ-(Z)_{g}-C(═NR¹³)—NR¹³R¹³, -Het-(CH₂)ₙ-(Z)_{g}-(CHOR⁸)ₘ-(Z)_{g}-C(═NR¹³)—NR¹³R¹³, —(CH₂)ₙ—NR¹²R¹², —O—(CH₂)ₘ—NR¹²R¹², —O—(CH₂)ₙ—NR¹²R¹², —O—(CH₂)ₘ(Z)_{g}R¹², —(CH₂)ₙNR¹¹R¹¹, —O—(CH₂)ₘNR¹¹R¹¹, —(CH₂)ₙ—N⊕—(R¹¹)₃, —O—(CH₂)ₘ—N⊕—(R¹¹)₃, —(CH₂)ₙ-(Z)_{g}-(CH₂)ₘ—NR¹⁰R¹⁰, —O—(CH₂)ₘ-(Z)_{g}-(CH₂)ₘ—NR¹⁰R¹⁰, —(CH₂CH₂O)ₘ—CH₂CH₂NR¹²R¹², —O—(CH₂CH₂O)ₘ—CH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰-(Z)_{g}-R¹⁰, —(CH₂)ₙ—(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g}-R¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂CH₂O)ₘ—R⁸, -(Het)-(CH₂CH₂O)ₘ—CH₂CH₂NR⁷R¹⁰, -(Het)-(CH₂)ₘ—C(═O)NR⁷R¹⁰, -(Het)-(CH₂)ₘ-(Z)_{g}-R⁷, -(Het)-(CH₂)ₘ—NR¹⁰—CH₂(CHOR⁸)(CHOR⁸)ₙ—CH₂OR⁸, -(Het)-(CH₂)ₘ—CO₂R⁷, -(Het)-(CH₂)ₘ—NR¹²R¹², -(Het)-(CH₂)ₙ—NR¹²R¹², -(Het)-(CH₂)ₘ-(Z)_{g}R¹², -(Het)-(CH₂)ₘNR¹¹R¹¹, -(Het)-(CH₂)ₘ—N⊕—(R¹¹)₃, -(Het)-(CH₂)ₘ-(Z)_{g}-(CH₂)ₙ, —NR¹⁰R¹⁰, -(Het)-(CH₂CH₂O)ₘ—CH₂CH₂NR¹²R¹², -(Het)-(CH₂)ₘ—(C═O)NR¹²R₁₂, -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, Link-(CH₂)ₙ-CAP, Link-(CH₂)ₙ(CHOR⁸)(CHOR⁸)ₙ-CAP, Link-(CH₂CH₂O)ₘ—CH₂-CAP, Link-(CH₂CH₂O)ₘ—CH₂CH₂-CAP, Link-(CH₂)ₙ-(Z)_{g}-CAP, Link-(CH₂)ₙ(Z)_{g}-(CH₂)ₘ-CAP, Link-(CH₂)ₙ—NR¹³—CH₂(CHOR⁸)(CHOR⁸)ₙ-CAP, Link-(CH₂)ₙ—(CHOR⁸)ₘCH₂—NR¹³-(Z)_{g}-CAP, Link-(CH₂)ₙNR¹³—(CH₂)ₘ(CHOR⁸)ₙCH₂NR¹³-(Z)_{g}-CAP, -Link-(CH₂)ₘ-(Z)_{g}-(CH₂)ₘ-CAP, Link-NH—C(═O)—NH—(CH₂)ₘ-CAP, Link-(CH₂)ₘ—C(═O)NR¹³—(CH₂)ₘ—C(═O)NR¹⁰R¹⁰, Link-(CH₂)ₘ—C(═O)NR¹³—(CH₂)ₘ-CAP, Link-(CH₂)ₘ—C(═O)NR¹¹R¹¹, Link-(CH₂)ₘ—C(═O)NR₁₂R₁₂, Link-(CH₂)ₙ-(Z)_{g}-(CH₂)ₘ-(Z)_{g}-CAP, Link-(Z)_{g}-(CH₂)ₘ-Het-(CH₂)ₘ-CAP, Link-(CH₂)ₙ—CR¹¹R¹¹-CAP, Link-(CH₂)ₙ(CHOR⁸)(CHOR⁸)ₙ-CR¹¹R¹¹-CAP, Link -(CH₂CH₂O)ₘ—CH₂-CR¹¹R¹¹-CAP, Link-(CH₂CH₂O)ₘ—CH₂CH₂-CR¹¹R¹¹-CAP, Link-(CH₂)ₙ-(Z)_{g}-CR¹¹R¹¹-CAP, Link-(CH₂)ₙ(Z)_{g}-(CH₂)ₘ—CR¹¹R¹¹-CAP, Link-(CH₂)ₙ—NR¹³-CH₂(CHOR⁸)(CHOR⁸)ₙ—CR¹¹R¹¹-CAP, Link-(CH₂)ₙ—(CHOR⁸)ₘCH₂—NR¹³-(Z)_{g} CR¹¹R¹¹ CAP, Link-(CH₂)ₙNR¹³—(CH₂)ₘ(CHOR⁸)ₙCH₂NR¹³-(Z)_{g}-CR¹¹R¹¹-CAP, Link-(CH₂)ₘ-(Z)_{g}-(CH₂)ₘ—CR¹¹R¹¹-CAP, Link NH—C(═O)—NH—(CH₂)ₘ—CR¹¹R¹¹-CAP, Link-(CH₂)ₘ—C(═O)NR¹³—(CH₂)ₘ—CR¹¹R¹¹-CAP, Link (CH₂)ₙ-(Z)_{g}-(CH₂)ₘ-(Z)_{g}-CR¹¹R¹¹-CAP, or Link-(Z)_{g}-(CH₂)ₘ-Het-(CH₂)ₘ—CR¹¹R¹¹-CAP;
each R⁶ is, independently, R⁵, —R⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁷, —(CH₂)ₙ—CO₂R⁷, —O—(CH₂)ₘ—CO₂R⁷, —OSO₃H, —O-glucuronide, —O-glucose, 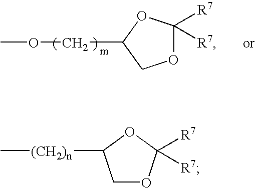
wherein when two R⁶ are —OR¹¹ and are located adjacent to each other on the aromatic carbocycle or aromatic heterocycle, the two OR¹¹ may form a methylenedioxy group;
each R⁷ is, independently, hydrogen, lower alkyl, phenyl, substituted phenyl or —CH₂(CHOR⁸ )ₘ—CH₂OR⁸;
each R⁸ is, independently, hydrogen, lower alkyl, —C(═O)—R¹¹, glucuronide, 2-tetrahydropyranyl, or 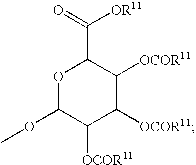
each R⁹ is, independently, —CO₂R⁷, —CON(R⁷)₂, —SO₂CH₃, —C(═O)R⁷, —CO₂R¹³, —CON(R¹³)₂, —SO₂CH₂R¹³, or —C(═O)R¹³;
each R¹⁰ is, independently, —H, —SO₂CH₃, —CO₂R⁷, —C(═O)NR⁷R⁹, —C(═O)R⁷, or —CH₂—(CHOH)ₙ—CH₂OH;
each Z is, independently, —(CHOH)—, —C(═O)—, —(CHNR⁷R¹⁰)—, —(C═NR¹⁰)—, —NR¹⁰—, —(CH₂)ₙ—, —(CHNR¹³R¹³)—, —(C═NR¹³)—, or —NR¹³—;
each R¹¹ is, independently, hydrogen, lower alkyl, phenyl lower alkyl or substituted phenyl lower alkyl;
each R¹² is, independently, —SO₂CH₃, —CO₂R⁷, —C(═O)NR¹³R⁷, —C(═O)R⁷, —CH₂(CHOH)ₙ—CH₂OH, —CO₂R¹³, —C(═O)NR¹³R¹³, or —C(═O)R¹³;
each R¹³ is, independently, R⁷, R¹⁰, —(CH₂)ₘ—NR⁷R¹⁰, —(CH₂)ₘ—NR⁷R⁷, —(CH₂)ₘ—NR¹¹R¹¹, —(CH₂)ₘ—(NR¹¹R¹¹R¹¹)⁺, —(CH₂)ₘ—(CHOR⁸)ₘ—(CH₂)ₘNR¹¹R¹¹, —(CH₂)ₘ—(CHOR⁸)ₘ—(CH₂)ₘNR⁷R¹⁰, —(CH₂)ₘ—NR¹⁰R¹⁰, —(CH₂)ₘ—(CHOR⁸)ₘ—(CH₂)ₘ—(NR¹¹R¹¹R¹¹)⁺, —(CH₂)ₘ—(CHOR⁸)ₘ—(CH₂)ₘNR⁷R⁷, 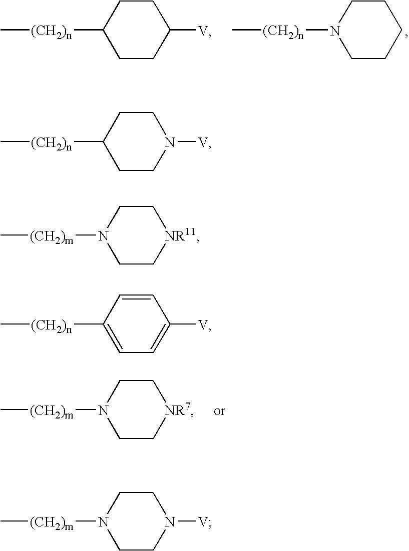
with the proviso that in the moiety —NR¹³R¹³, the two R¹³ along with the nitrogen to which they are attached may, optionally, form a ring selected from: 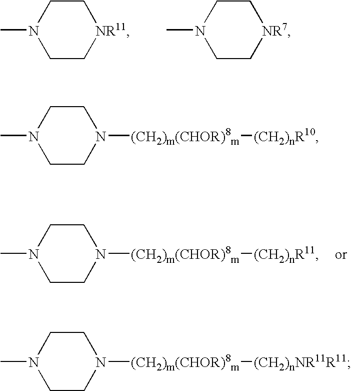
each V is, independently, —(CH₂)ₘ—NR⁷R¹⁰, —(CH₂)ₘ—NR⁷R⁷, —(CH₂)ₘ—(NR¹¹R¹¹R¹¹)⁺, —(CH₂)ₙ—(CHOR⁸)ₘ—(CH₂)ₘNR⁷R¹⁰, —(CH₂)ₙ—NR¹⁰R¹⁰—(CH₂)ₙ—(CHOR⁸)ₘ—(CH₂)ₘNR⁷R⁷, —(CH₂)ₙ—(CHOR⁸)ₘ—(CH₂)ₘ—(NR¹¹R¹¹R¹¹)⁺with the proviso that when V is attached directly to a nitrogen atom, then V can also be, independently, R⁷, R¹⁰, or (R¹¹)₂;
each R¹⁴ is, independently, H, R¹², —(CH₂)ₙ—SO₂CH₃, —(CH₂)ₙ—CO₂R¹³, —(CH₂)ₙ—C(═O)NR¹³R¹³, —(CH₂)ₙ—C(═O)R¹³, —(CH₂)ₙ—(CHOH)ₙ—CH₂OH, —NH—(CH₂)ₙ—SO₂CH₃, NH—(CH₂)ₙ—C(═O)R¹¹, NH—C(═O)—NH—C(═O)R¹¹, —C(═O)NR¹³R¹³, —OR¹¹, —NH—(CH₂)ₙ—R¹⁰, —Br, —Cl, —F, —I, SO₂NHR¹¹, —NHR¹³, —NH—C(═O)—NR¹³R¹³, —(CH₂)ₙ—NHR¹³, or —NH—(CH₂)ₙ—C(═O) R¹³;
each g is, independently, an integer from 1 to 6;
each m is, independently, an integer from 1 to 7;
each n is, independently, an integer from 0 to 7;
each -Het- is, independently, —N(R⁷)—, —N(R¹⁰)—, —S—, —SO—, —SO₂—; —O—, —SO₂NH—, —NHSO₂—, —NR⁷CO—, —CONR⁷—, —N(R¹³)—, —SO₂NR¹³—, —NR¹³CO—, or —CONR¹³—;
each Link is, independently, —O—, —(CH₂)ₙ—, —O(CH₂)ₘ—, —NR¹³—C(═O)—NR¹³—, —NR¹³—C(═O)—(CH₂)ₘ—, —C(═O)NR¹³—(CH₂)ₘ—, —(CH₂)ₙ-(Z)_{g}-(CH₂)ₙ—, —S—, —SO—, —SO₂—, —SO₂NR⁷—, —SO₂NR¹⁰—, or -Het-;
each CAP is, independently, thiazolidinedione, oxazolidinedione, -heteroaryl-C(═O)N R¹³R¹³, heteroaryl-W, —CN, —O—C(═S)NR¹³R¹³, -(Z)_{g}R¹³, —CR¹⁰((Z)_{g}R¹³)((Z)_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, a cyclic sugar or oligosaccharide, a cyclic amino sugar, oligosaccharide, —CR¹⁰(—(CH₂)ₘ—R⁹)(—(CH₂)ₘ—R⁹), —N(—(CH₂)ₘ—R⁹)(—(CH₂)ₘ—R⁹), —NR¹³(—(CH₂)ₘ—CO₂R¹³), 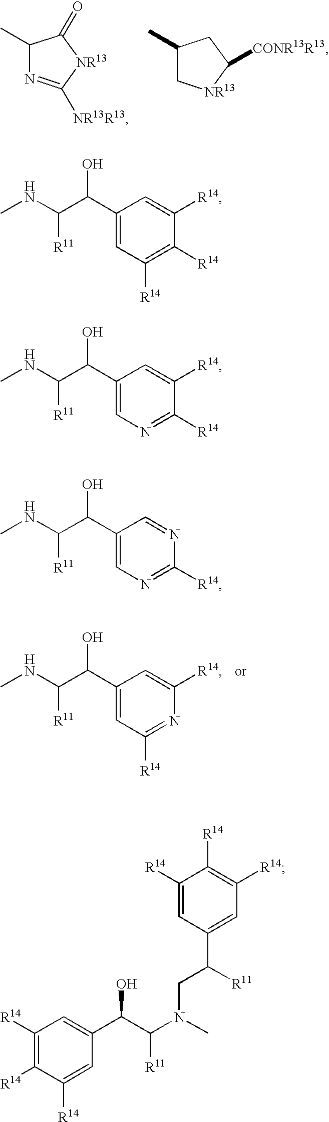
each Ar is, independently, phenyl, substituted phenyl, wherein the substituents of the substituted phenyl are 1-3 substituents independently selected from the group consisting of OH, OCH₃, NR¹³R¹³, Cl, F, and CH₃, or heteroaryl; and
each W is, independently, thiazolidinedione, oxazolidinedione, heteroaryl-C(═O)N R¹³R¹³, —CN, —O—C(═S)NR¹³R¹³, -(Z)_{g}R¹³, —CR¹⁰((Z)_{g}R¹³)((Z)_{g}R¹³), —C(═O)OAr, —C(═O)N R¹³Ar, imidazoline, tetrazole, tetrazole amide, —SO₂NHR¹³, SO₂NH—C(R¹³R¹³)-(Z)_{g}-R¹³, a cyclic sugar or oligosaccharide, a cyclic amino sugar, oligosaccharide, 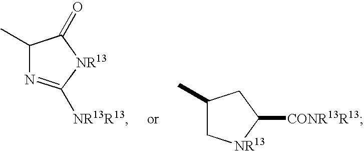
with the proviso that when any —CHOR⁸— or —CH₂OR⁸ groups are located 1,2- or 1,3- with respect to each other, the R⁸ groups may, optionally, be taken together to form a cyclic mono- or di-substituted 1,3-dioxane or 1,3-dioxolane.

2. The method of claim 1, which is a method of treating pain.

3. The method of claim 1, which is a method of treating ischemic pain due to cardiovascular disease.

4. The method of claim 1, which is a method of treating stroke-induced neural damage.

5. The method of claim 1, which is a method of treating pain due to arthritis.

6. The method of claim 1, which is a method of treating ischemic pain due to cancer.

7. The method of claim 1, which is a method of treating pain due to inflammation.

8. The method of claim 1, which is a method of treating pain due to infection.

9. The method of claim 1, which is a method of treating ischemic pain due to oropharengeal diseases or damage.

10. The method of claim 1, which is a method of treating ischemic pain due to traumatic injuries.

11. The method of claim 1, which is a method of treating acute and chronic cough.

12. The method of claim 1, which is a method of treating pain due to gastrointestinal disorders.

13. The method of claim 1, which is a method of treating central nervous system disorders.

14. The method of claim 1, which is a method of treating psychiatric diseases or manifestations.

15. The method of claim 1, wherein the compound of formula I is 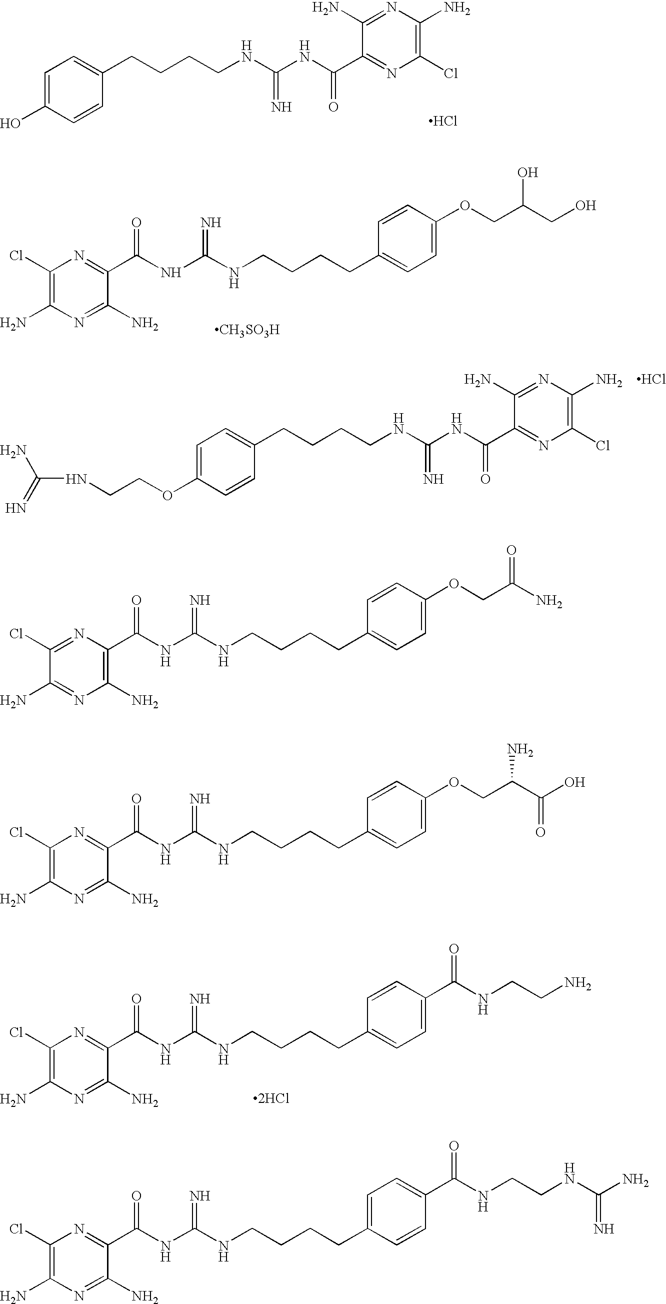 [chemical structure]
